(19) United States
(12) Patent Application Publication
(10) Pub. No.: US 2019/0008873 A1
(43) Pub. Date: Jan. 10, 2019

(54) NEUROACTIVE STEROID SOLUTIONS AND THEIR METHODS OF USE

(71) Applicant: Sage Therapeutics, Inc., Cambridge, MA (US)

(72) Inventors: Francesco G. Salituro, Marlborough, MA (US); Albert Jean Robichaud, Cambridge, MA (US); Paul Steven Watson, Carrboro, NC (US); William Hunke, Middletown, DE (US)

(21) Appl. No.: 15/737,676

(22) PCT Filed: Jun. 17, 2016
(86) PCT No.: PCT/US16/38195

§ 371 Date: Dec. 18, 2017

Related U.S. Application Data
(60) Provisional application No. 62/181,550, filed on Jun. 18, 2015.

(51) Int. Cl. A61K 31/573; A61K 47/69; A61K 47/12; A61K 9/00; A61K 9/08

(52) U.S. Cl. CPC A61K 31/573; A61K 47/6951; A61P 25/24; A61K 9/0019; A61K 9/08; A61K 47/12

(57) ABSTRACT

Provided herein are pharmaceutically acceptable aqueous solution comprising a neuroactive steroid, a sulfobutyl ether beta cyclodextrin and a buffer; wherein: the solution is a stable solution between a pH of about 3 and about 9, e.g., at room temperature, for at least 1, 2, 3, 4 weeks; 1, 2, 3, 4, 5, 6, 7, 8, 9, 10, 11, 12 months; 1, 2, 3 years or more; the buffer is present at a concentration of at least 0.1 mM; or the solution remains substantially free of impurities (e.g., the solution is substantially free of impurities at room temperature for at least 1, 2, 3, 4 weeks; 1, 2, 3, 4, 5, 6, 7, 8, 9, 10, 11, 12 months; 1, 2, 3 years or more).

### RELATED APPLICATIONS

This application claims priority under 35 U.S.C. § 119(e) to U.S. Provisional Application No. 62/181,550 filed Jun. 18, 2015, the entire contents of which are incorporated herein by reference.

### BACKGROUND

Homogeneous solutions (e.g., aqueous solutions) comprising a therapeutic agent, e.g., a neuroactive steroid described herein, enable administration to a human subject in need by various modes of administration (e.g., oral, parenteral (e.g., intravenous, intramuscular, subcutaneous) delivery). Neuroactive steroids are typically highly lipophilic compounds with low intrinsic water solubility. Particularly for intravenous administration, solutions are generally pH stable or chemically stable, preferably for an extended period of time.

### SUMMARY OF THE INVENTION

Provided herein is a pharmaceutically acceptable aqueous solution comprising (e.g., consisting essentially of, consisting of) a neuroactive steroid (e.g., allopregnanolone), a sulfobutyl ether beta cyclodextrin and a buffer; wherein: the solution is a stable solution between a pH of about 3 and about 9 (e.g., between about 5 and about 7, between about 5.5 and about 6.5), for at least 1, 2, 3, 4 weeks; 1, 2, 3, 4, 5, 6, 7, 8, 9, 10, 11, 12 months; 1, 2, 3 years or more.

In some embodiments, the solution is a stable solution between a pH of about 3 and about 9 (e.g., between about 5 and about 7, between about 5.5 and about 6.5), for at least 1, 2, 3, 4 weeks; 1, 2, 3, 4, 5, 6, 7, 8, 9, 10, 11, 12 months; 1, 2, 3 years or more at a temperature from about 2° C. to about 8° C.

In some embodiments, the solution is a stable solution between a pH of about 3 and about 9 (e.g., between about 5 and about 7, between about 5.5 and about 6.5), for at least 1, 2, 3, 4 weeks; 1, 2, 3, 4, 5, 6, 7, 8, 9, 10, 11, 12 months; 1, 2, 3 years or more at a temperature from about 0° C. to about 45° C. (e.g., between about 0° C. to about 30° C., between about 15° C. to about 25° C.).

Also provided herein is a pharmaceutically acceptable aqueous solution comprising (e.g., consisting essentially of, consisting of) a neuroactive steroid (e.g., allopregnanolone), a sulfobutyl ether beta cyclodextrin and a buffer; wherein: the buffer is present at a concentration of at least 0.1 mM (e.g., at least 0.5 mM, 1 mM, 2 mM, 5 mM, or 10 mM).

Also provided herein is a pharmaceutically acceptable aqueous solution comprising (e.g., consisting essentially of, consisting of) a neuroactive steroid (e.g., allopregnanolone), a sulfobutyl ether beta cyclodextrin and a buffer; wherein: the solution remains substantially free (e.g., meets product specifications of less than 3, 2, 1, 0.5, 0.3, 0.2, 0.1% w/w) of impurities (e.g., the solution is substantially free (e.g., meets product specifications of less than 3, 2, 1, 0.5, 0.3, 0.2, 0.1% w/w) of impurities at room temperature for at least 1, 2, 3, 4 weeks; 1, 2, 3, 4, 5, 6, 7, 8, 9, 10, 11, 12 months; 1, 2, 3 years or more). In some embodiments, the solution has at least 97% purity for at least 1, 2, 3, 4 weeks; 1, 2, 3, 4, 5, 6, 7, 8, 9, 10, 11, 12 months; 1, 2, 3 years or more). For example, the solution has 90-110 assay value for at least 1, 2, 3, 4 weeks; 1, 2, 3, 4, 5, 6, 7, 8, 9, 10, 11, 12 months; 1, 2, 3 years or more).

In some embodiments, the solution remains substantially free (e.g., less than 3, 2, 1, 0.5, 0.3, 0.2, 0.1% w/w) of impurities for at least 1, 2, 3, 4 weeks; 1, 2, 3, 4, 5, 6, 7, 8, 9, 10, 11, 12 months; 1, 2, 3 years or more at a temperature from about 2° C. to about 8° C.

In some embodiments, the solution remains substantially free (e.g., less than 3, 2, 1, 0.5, 0.3, 0.2, 0.1% w/w) of impurities for at least 1, 2, 3, 4 weeks; 1, 2, 3, 4, 5, 6, 7, 8, 9, 10, 11, 12 months; 1, 2, 3 years or more at a temperature from about 0° C. to about 45° C. (e.g., between about 0° C. to about 30° C., between about 15° C. to about 25° C.).

In some embodiments, the buffer in the solution is present at a concentration of from about 5 to 10 mM. In some embodiments, the buffer in the solution is present at a concentration of from about 0.1 to about 4 mM. In some embodiments, the buffer in the solution is present at a concentration of about 0.1, about 0.5, about 1.67, or about 3.3 mM.

In some embodiments, the solution further comprises a diluent.

In some embodiments, the solution is suitable for parenteral use.

In some embodiments, the solution is homogeneous.

In some embodiments, the neuroactive steroid is selected from pregnanolone, ganaxolone, alphadalone, alphaxalone, and allopregnanolone. In some embodiments, the neuroactive steroid is ganaxolone. In some embodiments, the neuroactive steroid is allopregnanolone.

In some embodiments, the neuroactive steroid is an estrol.

In some embodiments, the assay of the neuroactive steroid decreases less than 10% during storage for 1, 2, 3, 4, 5, 6, 7 days; 1, 2, 3, 4, 5, 6 months or more or 1, 2, 3 years or more at room temperature (e.g., 23+/−2° C.).

In some embodiments, the assay of the neuroactive steroid decreases less than 10% during storage for 1, 2, 3, 4, 5, 6, 7 days; 1, 2, 3, 4, 5, 6 months or more or 1, 2, 3 years or more at about 2 to about 8° C.

In some embodiments, the assay of the neuroactive steroid decreases less than 10% during storage for at least 10, 15, 20, 25, 30, 40, 45 minutes or more at about 110 to about 130° C. (e.g., about 110 to about 125° C., e.g., 122+/−2° C.).

In some embodiments, the solution has an assay value of 100+/−10%.

In some embodiments, the solution is chemically stable. In some embodiments, the solution is physically stable. In some embodiments, the solution is pH-stable.

In some embodiments, the solution includes less than 0.5, 0.4, 0.3, 0.2, or 0.1% w/w of a degradant of a neuroactive steroid (e.g., allopregnanolone). In some embodiments, the degradant is an oxidative product of the neuroactive steroid (e.g., oxidative product of allopregnanolone, 136). In some embodiments, the degradant is a racemate or epimer of the neuroactive steroid (e.g., epimer product of allopregnanolone, 1269). In some embodiments, the amount of degradant of the neuroactive steroid (e.g., racemate or epimer or oxidative product of the neuroactive steroid) present in the solution is substantially similar (e.g., meets product specifications of +/−0.1, 0.2, 0.5, 1, 2% w/w %) for 1, 2, 3, 4, 5, 6, 7 days or more; 1, 2, 3, 4 weeks or more; 1, 2, 3, 4, 5, 6, 7, 8, 9, 10, 11, 12 months or more; 1, 2, 3 years or more. In some embodiments, the amount of degradant of the neuroactive steroid present in the solution is less than 0.1% w/w for 1, 2, 3, 4, 5, 6, 7 days or more; 1, 2, 3, 4 weeks or more; 1, 2, 3, 4, 5, 6, 7, 8, 9, 10, 11, 12 months or more; 1, 2, 3 years or more.

In some embodiments, the pH of the solution is substantially similar (e.g., meets product specifications; the pH is +/−1.2, 1, 0.8, 0.5, 0.3 or less) for 1, 2, 3, 4, 5, 6, 7 days or more; 1, 2, 3, 4 weeks or more; 1, 2, 3, 4, 5, 6, 7, 8, 9, 10, 11, 12 months or more; 1, 2, 3 years or more.

In some embodiments, the pH of the solution is from about 3 and about 9 (e.g., between about 5 and about 7, between about 5.5 and about 6.5) for 1, 2, 3, 4, 5, 6, 7 days or more; 1, 2, 3, 4 weeks or more; 1, 2, 3, 4, 5, 6, 7, 8, 9, 10, 11, 12 months or more; 1, 2, 3 years or more.

In some embodiments, the solution is at between 3° C. and 37° C. In some embodiments, the solution is at between 0° C. and 45° C. (e.g., between 0° C. and 30° C., e.g., between 15° C. and 25° C.). In some embodiments, the solution is at room temperature (e.g., 25° C.).

In some embodiments, the buffer is selected from an acidic, basic, or neutral buffer. In some embodiments, the buffer is selected from an acidic or neutral buffer. In some embodiments, the buffer has a pKa of about 2 to about 9. In some embodiments, the buffer comprises a monoprotic acid. In some embodiments, the buffer comprises a polyprotic acid (e.g., citrate). In some embodiments, the buffer is selected from the group consisting of citrate, phosphate, acetate, lactate, gluconate, malate, succinate, Tris, histidine, and tartrate and mixtures thereof.

In some embodiments, the buffer is citrate buffer. In some embodiments, the citrate buffer has a pH from about 3 to about 8 (e.g., about 4.5 to about 7.0, about 5.5 to about 6.5, about 5.0 to about 6.0).

In some embodiments, the buffer is phosphate buffer. In some embodiments, the phosphate buffer has a pH from about 1 to about 9 (e.g., about 4.5 to about 7.0, about 5.5 to about 6.5, about 5.0 to about 6.0).

In some embodiments, the buffer is a solution of one or more substances (e.g., a salt of a weak acid and a weak base; a mixture of a weak acid and a salt of the weak acid with a strong base).

In some embodiments, the buffer is selected from 4-2-hydroxyethyl-1-piperazineethanesulfonic acid (HEPES), 2-{[tris(hydroxymethyl)methyl]amino}ethanesulfonic acid (TES), 3-(N-morpholino)propanesulfonic acid (MOPS), piperazine-N,N′-bis(2-ethanesulfonic acid) (PIPES), dimethylarsinic acid (cacodylate), Citrate (e.g., saline sodium citrate), 2-(N-morpholino)ethanesulfonic acid (MES), phosphate (e.g., PBS, D-PBS), succinate (i.e., 2(R)-2-(methylamino)succinic acid), acetate, dimethylglutarate, maleate, imidazole, N-(2-Acetamido)-2-aminoethanesulfonic acid (ACES), N,N-bis(2-hydroxyethyl)-2-aminoethanesulfonic acid (BES), Bicine, Bis-Tris, Borate, N-cyclohexyl-3-aminopropanesulfonic acid (CAPS), Glycine, 3-[4-(2-Hydroxyethyl)-1-piperazinyl]propanesulfonic acid (HEPPS or EPPS), N-[Tris(hydroxymethyl)methyl]-3-aminopropanesulfonic acid, [(2-Hydroxy-1,1-bis(hydroxymethyl)ethyl)amino]-1-propanesulfonic acid (TAPS), Tricine, Tris, Tris Base, Tris Buffer, Tris-Glycine, Tris-HCl, collidine, veronal acetate, N-(2-Acetamido)iminodiacetic acid; N-(Carbamoylmethyl)iminodiacetic acid (ADA), 13-Hydroxy-4-morpholinepropanesulfonic acid, 3-Morpholino-2-hydroxypropanesulfonic acid (MOPSO), cholamine chloride, 3-(N,N-Bis[2-hydroxyethyl]amino)-2-hydroxypropanesulfonic acid (DIPSO), acetamidoglycine, 3-{[1,3-Dihydroxy-2-(hydroxymethyl)-2-propanyl]amino}-2-hydroxy-1-propanesulfonic acid (TAPSO), Piperazine-N,N′-bis(2-hydroxypropanesulfonic acid) (POPSO), N-(2-Hydroxyethyl)piperazine-N′-(2-hydroxypropanesulfonic acid) (HEPPSO), N-cycloxhexyl-2-aminoethanesulfonic acid (CHES), 2-amino-methyl-1,3-proponediol (AMPd), and glycinamide. In some embodiments, the buffer comprises a piperazine (e.g., PIPES, HEPES, POPSO, EPPS).

In some embodiments, the buffer comprises a non-metal complexing compound (e.g., MES, MOPS, PIPES).

In some embodiments, the buffer is at a pH suitable for injection (e.g., safe, tolerable, non-irritating).

In some embodiments, the buffer is within its range of effective buffer capacity.

In some embodiments, the buffer is citrate. In some embodiments, the citrate buffer is present at a concentration of about 1 to about 100 mM or more. In some embodiments, the citrate buffer is present at a concentration of 5, 10, 20, 50, 100 mM or more.

In some embodiments, the buffer is phosphate. In some embodiments, the phosphate buffer is present at a concentration of about 1 to about 100 mM or more. In some embodiments, the phosphate buffer is present at a concentration of 5, 10, 20, 50, 100 mM or more.

In some embodiments, the pH of the solution is about 3 to about 9 (e.g., preferably about 5 to about 9, about 4.5 to about 7.0, about 5.0 to about 6.5).

In some embodiments, the neuroactive steroid is present at 0.1, 0.5, 1, 1.25, 2.5, 3.75, 5, 6.25, 7.5, 8, 9, or 10 mg/mL or more. In some embodiments, the neuroactive steroid is formulated with 2.5, 5, 6, 7.5, 10, 15, 20, 30% w/v or more of sulfobutylether-β-cyclodextrin.

In some embodiments, the molar ratio of neuroactive steroid to sulfoalkylether-βcyclodextrin is about 1:1, 1:2, 1:3, 1:4, 1:5, 1:6, 1:7, 1:8, 1:9, 1:10, 1:20: 1:30, 1:50, 1:75, 1:100, 1:120 or more. In some embodiments, the molar ratio of neuroactive steroid to sulfoalkylether-βcyclodextrin is about 0.1, 0.05, 0.03, 0.02, 0.01, 0.008, 0.005 or less. In some embodiments, the neuroactive steroid is allopregnanolone. In some embodiments, the molar ratio of allopregnanolone to sulfoalkylether-βcyclodextrin is about 1:1, 1:2, 1:3, 1:4, 1:5, 1:6, 1:7, 1:8, 1:9, 1:10, 1:20: 1:30, 1:50, 1:75. In some embodiments, the molar ratio of allopregnanolone to sulfoalkylether-βcyclodextrin is about 1:1, 1:2, 1:3, 1:4, 1:5, 1:6, 1:7, 1:8, 1:9, 1:10, 1:20. In some embodiments, the molar ratio of allopregnanolone to sulfoalkylether-βcyclodextrin is about 1:1 to about 1:60 (e.g., about 1:1 to about 1:20, about 1:1 to about 1:15). In some embodiments, the molar ratio of allopregnanolone to sulfoalkylether-βcyclodextrin is about 1:3 to about 1:20 (e.g., about 1:5 to about 1:10). In some embodiments, the solution additionally comprises a surfactant.

In some embodiments, the solution additionally comprises a chelating agent.

In some embodiments, the solution additionally comprises a preservative.

In some embodiments, the solution additionally comprises a isotonizing agent. In some embodiments, the isotonizing agent is present in an amount to obtain isotonicity.

In some embodiments, the solution is sterilized by heat treatment.

In an aspect, provided is a pharmaceutically acceptable aqueous solution comprising (e.g., consisting essentially of, consisting of) a neuroactive steroid, a sulfobutyl ether beta cyclodextrin and a buffer; the composition comprising less than 3, 2, 1, 0.5, 0.3, 0.2, 0.1% w/w) of impurities (e.g., the solution is substantially free (e.g., less than 3, 2, 1, 0.5, 0.3, 0.2, 0.1% w/w) of impurities for at least 1, 2, 3, 4 weeks; 1, 2, 3, 4, 5, 6, 7, 8, 9, 10, 11, 12 months; 1, 2, 3 years or more).

In an aspect, provided is a method for preparing a stable solution comprising allopregnanolone, the method comprising contacting allopregnanolone with a pharmaceutically acceptable aqueous solution comprising (e.g., consisting essentially of, consisting of) a sulfobutyl ether beta cyclodextrin and a buffer.

In some embodiments, the solution is at between about 0° C. to about 60° C. (e.g., between about 20° C. to about 50° C., between about 35° C. to about 45° C.). In some embodiments, the solution is at room temperature (e.g., 35-45° C.).

In some embodiments, the solution is chemically stable.

In some embodiments, the solution is autoclaved (e.g., subjected to cycles of heat sterilization, e.g., subjected to at least 10 (e.g., at least 15, 20, 30, 40 minutes) of heat (e.g., from 110 to 150° C. (e.g., 121 to 123° C.)). In some embodiments, the solution is at from 110 to 150° C. (e.g., 121 to 123° C.).

In some embodiments, the amount of degradant of the neuroactive steroid present in the solution is less than 0.1% w/w for 1, 2, 3, 4, 5, 6, 7 days or more; 1, 2, 3, 4 weeks or more; 1, 2, 3, 4, 5, 6, 7, 8, 9, 10, 11, 12 months or more; 1, 2, 3 years or more.

In one aspect, provided herein is a pharmaceutically acceptable aqueous solution comprising (e.g., consisting essentially of, consisting of) a neuroactive steroid (e.g., allopregnanolone), a sulfobutyl ether beta cyclodextrin and a buffer; wherein: the solution is a stable solution between a pH of about 3 and about 9 (e.g., between about 5 and about 7, between about 5.5 and about 6.5), for at least 5 minutes, e.g., at least 10, 15, 20, 25, 30, 35, 40, 45, 50, 55 or 60 minutes or more at a temperature from about 120° C. to about 124° C.; or the buffer is present at a concentration of at least 0.1 mM; or the solution remains substantially free (e.g., meets product specifications of less than 3, 2, 1, 0.5, 0.3, 0.2, 0.1% w/w) of impurities for at least 5 minutes, e.g., at least 10, 15, 20, 25, 30, 35, 40, 45, 50, 55 or 60 minutes or more at a temperature from about 120° C. to about 124° C.

In one aspect, provided herein is a method of parenteral administration, the method comprising mixing a first solution comprising allopregnanolone (e.g., a solution described herein) with a diluent (e.g., water or injection or saline solution) to provide a diluted solution; and parenterally administering the diluted solution to a subject. In some embodiments, the first solution is diluted with two parts diluent to one part first solution. In some embodiments, the first solution is diluted with nine parts diluent to one part first solution.

In one aspect, provided herein is a method of preparing an aqueous solution comprising a neuroactive steroid, a sulfoalkyl ether beta cyclodextrin (e.g., sulfobutyl ether beta cyclodextrin or sulfobutylether-β-cyclodextrin), and a buffer, wherein the solution is mixed (e.g., by high-shear homogenization) to provide a solution substantially free (e.g., less than about 1, 0.5, 0.2, 0.1% w/v) of solids (e.g., free of any solid with a particle size of 0.22, 0.45, 1 micron or greater in diameter).

In some embodiments, the solution is mixed with a suitable mixing device or method. In some embodiments, the mixing device is a high shear impeller mixer, rotor stator mixer, homogenizer, ultrasonic device, or microfluidizer.

In some embodiments, the rotor stator mixer spins at 2,000 to 18,000 rpm. In some embodiments, the homogenizer functions at 1000 to 5000 psi.

In some embodiments, the solution is mixed by suitable high-shear mixing device such as rotor/stator device, a homogenizer, microfluidizer or sonication device. In some embodiments, the high shear mixing device (e.g., a rotor/stator, homogenizer, microfluidizer or sonication device uses inline high shear assemblies).

In some embodiments, the method is used for a suitable period of time to achieve solubilization (e.g., at least 15, 30, 60 or more minutes).

In some embodiments, the solution is diluted with a diluent, e.g., to produce an admixture.

In one aspect, provided herein is a closed container comprising a neuroactive steroid, a sulfoalkyl ether beta cyclodextrin (e.g., sulfobutyl ether beta cyclodextrin or sulfobutylether-β-cyclodextrin), and a buffer; additionally comprising a gaseous layer substantially comprising (e.g., comprising more than 90, 91, 92, 93, 94, 95, 96, 97, 98, 99, 99.1, 99.5, 99.98, 99.99% of an inert gas (e.g., nitrogen, argon).

In some embodiments, the gaseous layer comprises less than 21, 20, 17, 15, 12, 10, 8, 5, 3, 1, 0.5, 0.2, 0.1, 0.05% oxygen gas (e.g., free of oxygen gas).

In some embodiments, the container comprises a vial, stopper, or an overseal.

In some embodiments, the container is a prefilled syringe. In some embodiments, the container is a glass container. In some embodiments, the container is a plastic container. In some embodiments, the plastic container and low oxygen levels are provided by an overwrap (e.g aluminum laminate pouch).

In one aspect, provided herein is a method of treating a subject (e.g., a subject suffering from a disease or disorder described herein (e.g., depression (e.g., postpartum depression), the method comprising administering an aqueous solution or admixture described herein, thereby treating a subject.

In one aspect, provided herein is a method of treating a subject (e.g., a subject suffering from a disease or disorder described herein (e.g., depression (e.g., postpartum depression), the method comprising administering one part of an aqueous solution described herein, per two parts of a diluents described herein (e.g., WFI), thereby treating a subject.

In one aspect, provided herein is a method of treating a subject (e.g., a subject suffering from a disease or disorder described herein (e.g., depression (e.g., postpartum depression), the method comprising administering one part of an aqueous solution described herein, per nine parts of a diluents described herein (e.g., WFI), thereby treating a subject.

### DETAILED DESCRIPTION OF FIGURES

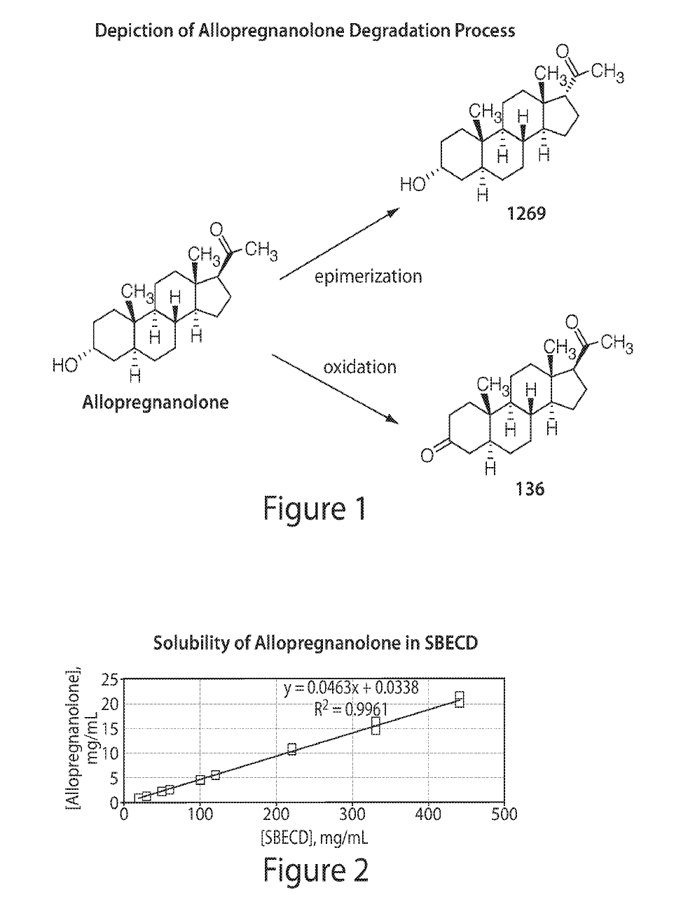

FIG. 1. Depiction of Allopregnanolone Degradation Processes

FIG. 2. Depiction of Allopregnanolone Solubility in SBECD

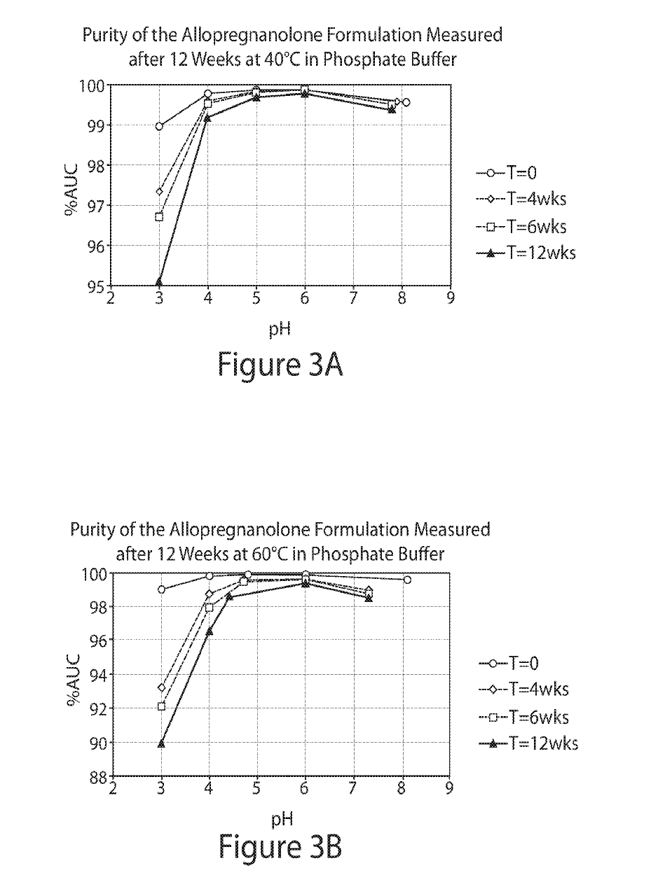

FIG. 3. Stability of Allopregnanolone in Phosphate Buffer at time=0, 4, 6, and 12 weeks (A) Area Under Curve at 40° C.; (B) Area Under Curve at 60° C.

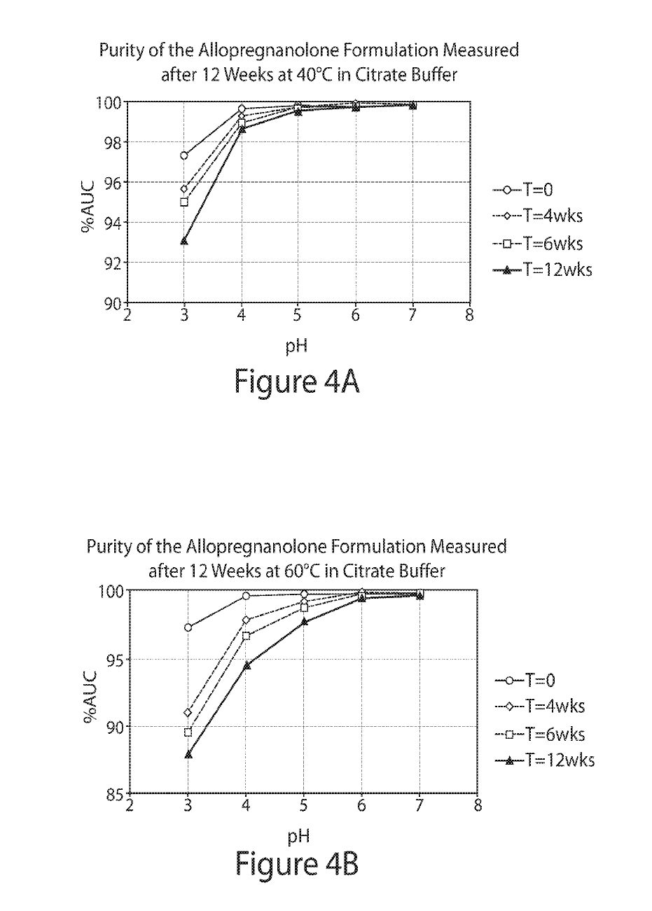

FIG. 4. Stability of Allopregnanolone in Citrate Buffer at time=0, 4, 6, and 12 weeks (A) Area Under Curve at 40° C.; (B) Area Under Curve at 60° C.

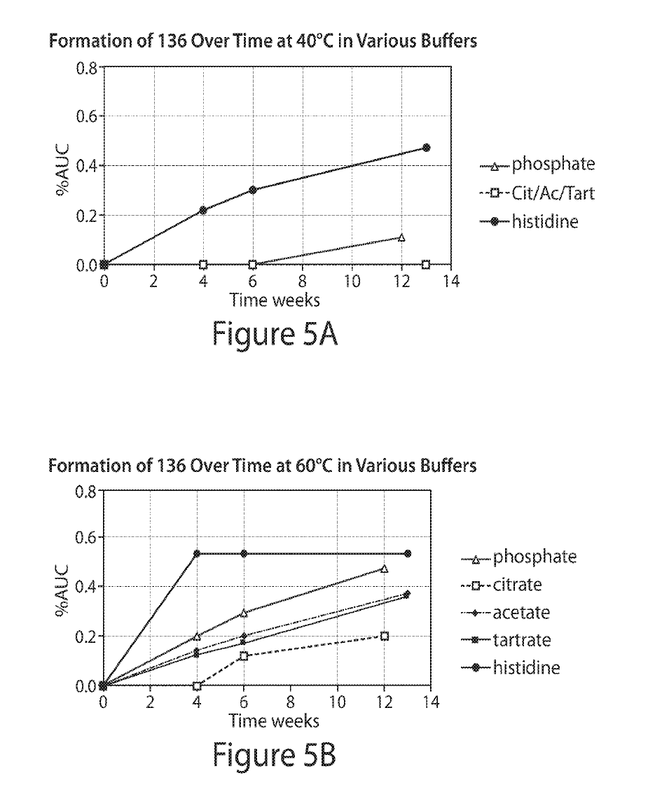

FIG. 5. Formation of 136 Over Time in Various Buffers (A) at 40° C.; (B) at 60° C.

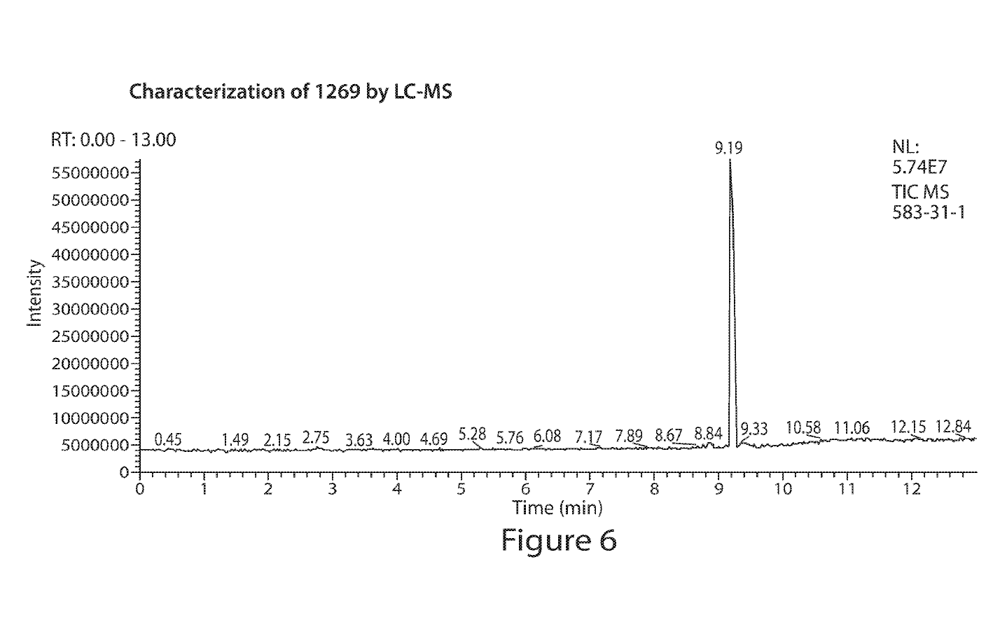

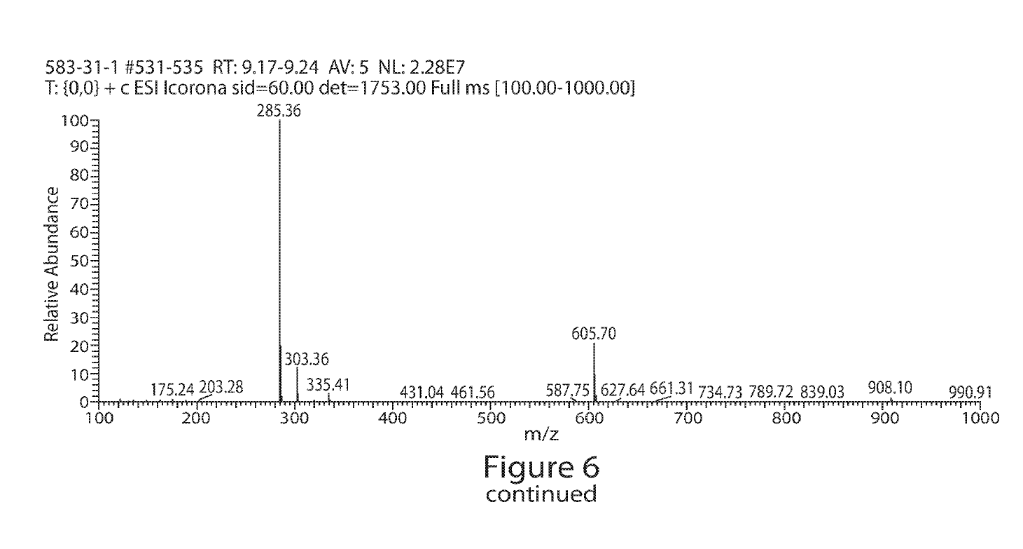

FIG. 6. Exemplary LC-MS Characterization of 1269

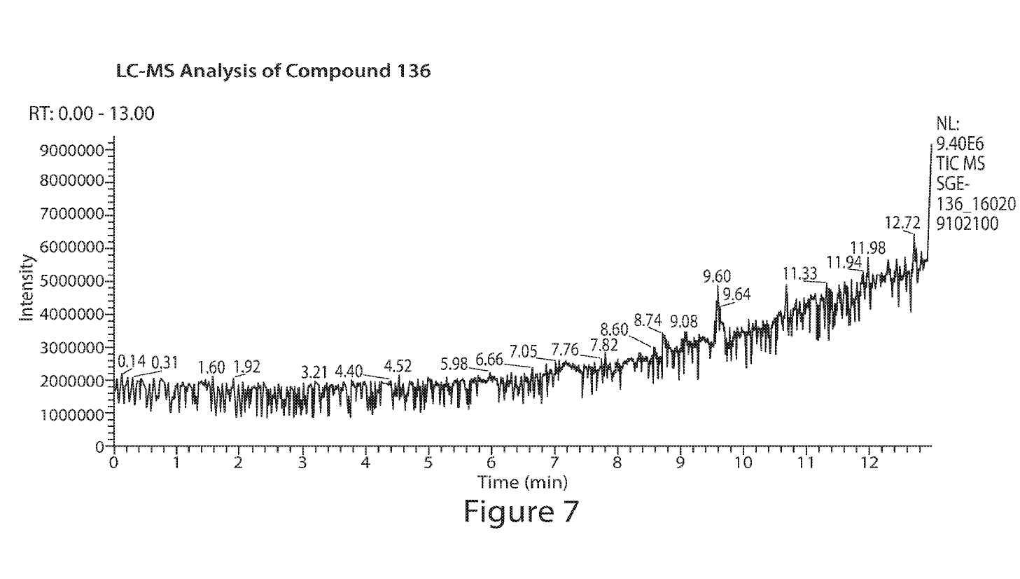

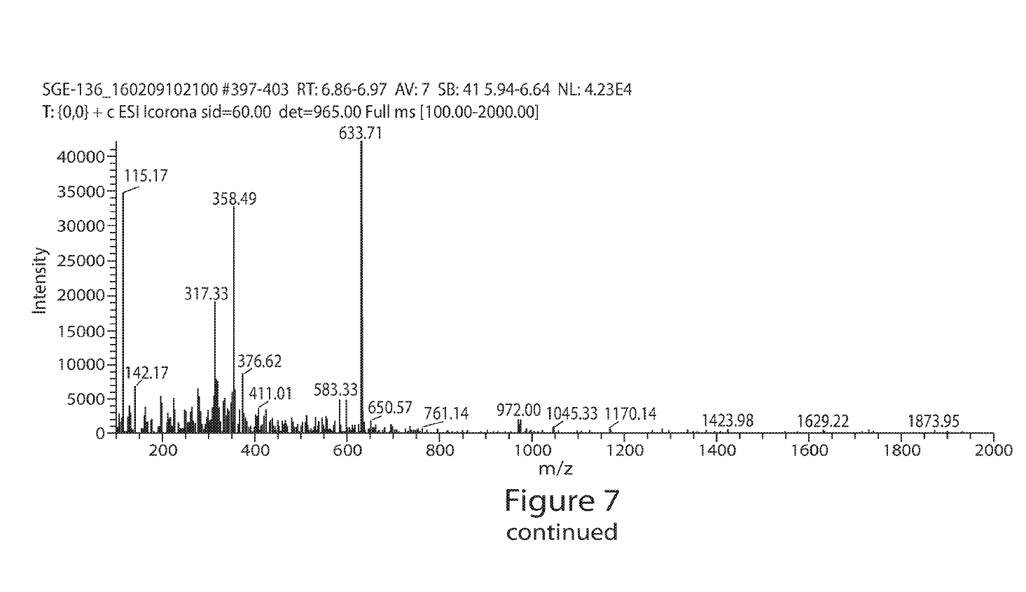

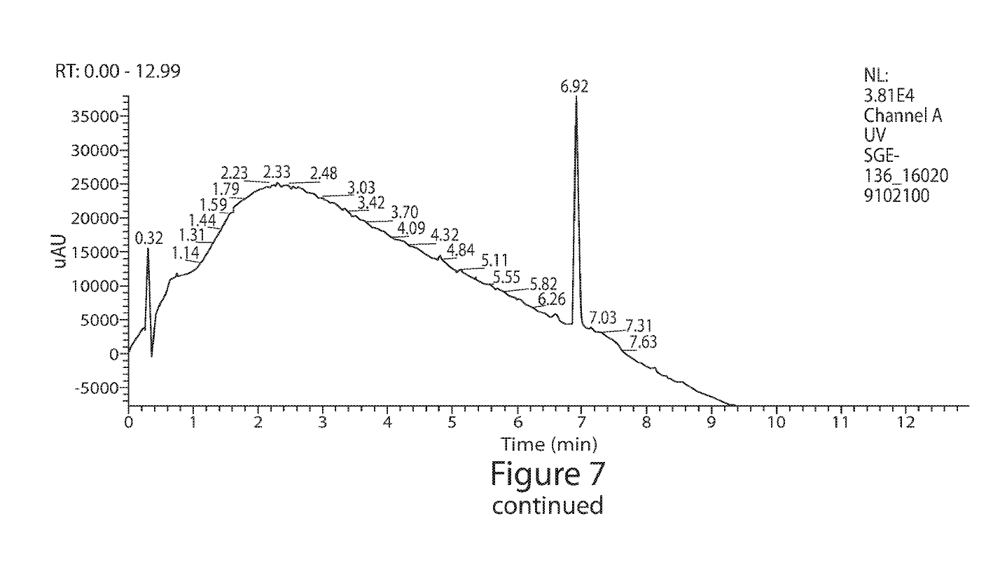

FIG. 7. Exemplary LC-MS Characterization of 136

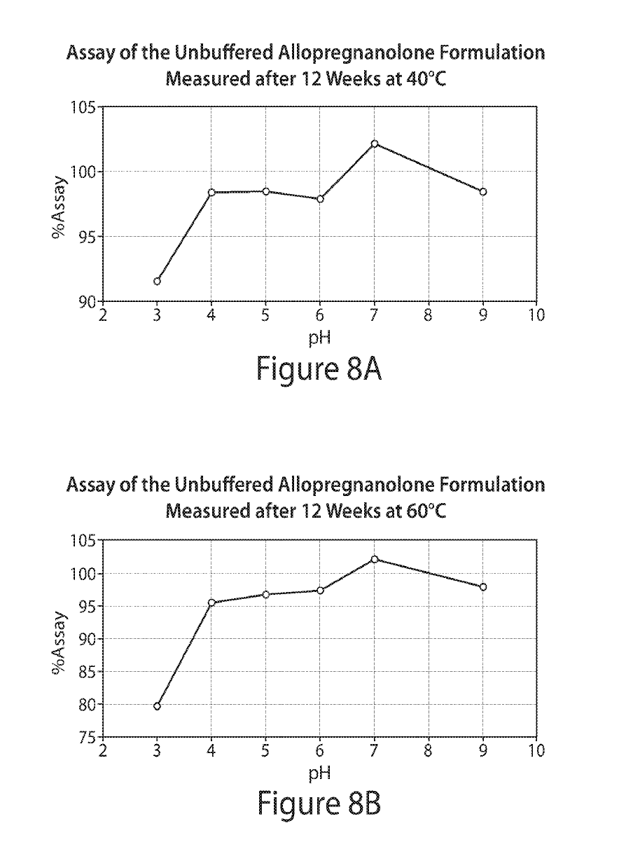

FIG. 8. Assay of Unbuffered Allopregnanolone Formulation Measured 12 Weeks at (A) 40° C. and (B) 60° C.

### DETAILED DESCRIPTION OF CERTAIN EMBODIMENTS OF THE INVENTION

Described herein are aqueous solutions or admixtures comprising a neuroactive steroid, a cyclodextrin, and a buffer; methods of their use and administration, methods for their preparation, and containers comprising the solutions or admixtures.

### Definitions

As used herein, the terms “stabilized” and “stable” aqueous solution described herein (e.g., an aqueous solution comprising a neuroactive steroid) refer to a solution that is “chemically stable” and “physically stable.” A solution comprising a neuroactive steroid is chemically stable if the neuroactive steroid does not undergo chemical transformation or degradation (e.g., racemization, epimerization, oxidation). For example, a chemically stable neuroactive steroid, e.g., in solution, will not racemize or epimerize (e.g., at susceptible positions (e.g., racemized or epimerized at the C17-position in a neuroactive steroid)) or oxidize (e.g., at susceptible positions (e.g., oxidized at the C3-position of a neuroactive steroid)) or reduce (e.g., at susceptible positions (e.g., reduced at the C21-position of a neuroactive steroid), e.g., after a period of time (e.g., for 1, 2, 3, 4, 5, 6, 8, 10, 12, 14, 16, 18, 20, 24 hours or more; 1, 2, 3, 4, 5, 6, 7 days or more; 1, 2, 3, 4 weeks or more; 1, 2, 3, 4, 5, 6, 8, 10, 12 months or more; 1, 2, 3, 4, 5 years or more) or at temperatures (e.g., ambient or elevated). As used herein, “pH-stable” refers to a solution in which the pH of the solution is substantially similar (e.g., +/−1.2, 1, 0.8, 0.5, 0.3 or less) for 1, 2, 3, 4, 5, 6, 7 days or more; 1, 2, 3, 4 weeks or more; 1, 2, 3, 4, 5, 6, 7, 8, 9, 10, 11, 12 months or more; 1, 2, 3, 4, 5 years or more, e.g., for at least 1, 2, 3, 4, 5, 6, 7, 8 weeks; 1, 2, 3, 4, 5, 6, 7, 8, 9, 10, 11, 12 months or more; 1, 2, 3, 4, 5 years or more. A solution comprising a neuroactive steroid is “physically stable” if the solution does not undergo physical changes, such as changes in color or the level of particulates, for example, after a period of time or at various temperatures. For example, a stable aqueous solution comprising a neuroactive steroid is chemically stable and physically stable under manufacturing (e.g., preparing; compounding, filling, labelling and sterilization), transportation, or storage conditions.

“Assay”, as used herein, refers to a specific, stability-indicating procedure that determines the content of the drug substance. For example, assay can be a chromagraphic method (e.g., HPLC) involving use of a reference standard.

“Impurities”, as used herein, refers to organic and inorganic impurities and residual solvents. For example, impurities refers to racemized or epimerized (e.g., at susceptible positions (e.g., racemized or epimerized at the C17-position in a neuroactive steroid)) or oxidized (e.g., at susceptible positions (e.g., oxidized at the C3-position of a neuroactive steroid)) or reduced (e.g., at susceptible positions (e.g., reduced at the C21-position of a neuroactive steroid), neuroactive steroid. A solution is free of impurities when it contains less than 3, 2, 1, 0.5, 0.3, 0.2, or 0.1% w/w impurities.

“Purity”, as used herein, refers to the absence of impurities, for example in a solution or composition, relative to its parent (e.g., at time=0).

“Sterilization”, as used herein, refers to aseptic fill (e.g., aseptic sterilization) or terminal sterilization.

### Solutions

The aqueous solutions or admixtures described herein comprise a neuroactive steroid. Neuroactive steroids are typically highly lipophilic compounds with low intrinsic water solubility. Cyclodextrins, e.g., cyclodextrins as described herein, may promote stabilization of compounds, e.g., neuroactive steroid compounds. It was unexpectedly found that certain unbuffered neuroactive steroid solutions comprising sulfobutylether-β-cyclodextrin were not pH stable. For example, the pH of the solutions (e.g., the unbuffered solutions) is between about 3 to about 9, e.g., between about 5 to about 8, e.g., between about 5.5 to about 7.5. Furthermore, the pH of the solutions (e.g., the unbuffered solutions), was found to drift (e.g., the pH did not remain between a desired pH range). It was found that certain buffers were well suited for combined use with unbuffered neuroactive steroid solutions comprising sulfobutylether-β-cyclodextrin, e.g., in clinical settings, because the pH of the solution or admixture does not change (e.g., the pH remained between 5.5 and 7.5). It was unexpectedly found that certain buffered solutions or admixtures were more stable than certain unbuffered solutions when stored for 1, 2, 3, 4, 5, 6 or more months at temperatures from 4 to 40° C. Moreover, it was surprisingly found that certain buffered solutions or admixtures described herein are stable (e.g., physically and chemically stable), e.g., at high temperatures (e.g., 121° C.) for short periods of time, to sterilization processes (e.g., sterilization processes described herein). For example, certain buffered solutions or admixtures described herein are stable (e.g., physically and chemically stable) at high temperatures (e.g., 121° C.) for 10, 20, 30, 40, 50, 60, 70, 80, 90 minutes or more. Further, certain buffered neuroactive steroid solutions or admixtures described herein were unexpectedly found to be less susceptible to the formation of impurities over a range of temperatures and times. For example, certain buffered neuroactive steroid solutions or admixtures may have a lower content of impurities (e.g., 2% w/v or lower) than certain unbuffered neuroactive steroid solutions over a range of temperature or storage times.

Certain buffered neuroactive steroid solutions or admixtures described herein are also stable (e.g., chemically and physically stable) for 1, 2, 3, 4, 5, 6, 8, 10, 12, 14, 16, 18, 20, 24 hours or more; 1, 2, 3, 4, 5, 6, 7 days or more; 1, 2, 3, 4 weeks or more; 1, 2, 3, 4, 5, 6, 8, 10, 12 months or more; 1, 2, 3, 4, 5 years or more. Certain buffered neuroactive steroid solutions or admixtures are stable (e.g., pH-stable, chemically stable) at between about 3 to about 125° C. In some embodiments, the buffered neuroactive steroid solutions or admixtures are stable at between about 3 to about 6° C. In some embodiments, the buffered neuroactive steroid solutions or admixtures are stable at about 4° C. In some embodiments, the buffered neuroactive steroid solutions or admixtures are stable at between about 20 to about 40° C. In some embodiments, the buffered neuroactive steroid solutions or admixtures are stable at room (e.g., ambient) temperature. In some embodiments, the buffered neuroactive steroid solutions or admixtures are stable at about 25° C. In some embodiments, the buffered neuroactive steroid solutions or admixtures are stable at about 37° C. In some embodiments, the buffered neuroactive steroid solutions or admixtures are stable at between about 115 to about 125° C., e.g., for several minutes (e.g., 10, 20, 30, 40, 50, 60, 70, 80, 90 minutes or more, for several hours (e.g., 1, 2, 3 hours or more). In some embodiments, the buffered neuroactive steroid solutions or admixtures are stable at autoclave temperature. In some embodiments, the buffered neuroactive steroid solutions or admixtures are stable at about 121° C.

In some embodiments, the buffered neuroactive steroid solutions or admixtures described herein are stable at temperatures ranging from about 20 to 30° C. for at least 1, 2, 3, 4, 5, 6, 7, 8 weeks; 1, 2, 3, 4, 5, 6, 7, 8, 9, 10, 11, 12 months or more; 1, 2, 3, 4, 5 years or more).

In some embodiments, the buffered neuroactive steroid solutions or admixtures described herein are stable at temperatures ranging from about 2 to 8° C. for at least 1, 2, 3, 4, 5, 6, 7, 8 weeks; 1, 2, 3, 4, 5, 6, 7, 8, 9, 10, 11, 12 months or more; 1, 2, 3, 4, 5 years or more).

In some embodiments, the buffered neuroactive steroid solutions or admixtures described herein are prepared for injection into a subject. As such they will be prepared by methods designed to ensure that they are sterile, and free of pyrogens, particulate matter, and other contaminents, and, where appropriate contain inhibitors of the growth of microorganisms. As such the buffered neuroactive steroid solutions or admixtures will be essentially free of visible solid particles. In some embodiments, the buffered neuroactive steroid solutions or admixtures described herein may be filtered. In some embodiments, the buffered neuroactive steroid solutions or admixtures described herein can be sterilized (e.g., sterilized by filtration (e.g., filtered through 0.45 and 0.22 micron filters), by heat (e.g., steam sterilization at 121° C., or by irradiation, e.g., gamma irradiation). In some embodiments, the sterilized buffered neuroactive steroid solutions or admixtures do not comprise higher levels of impurities (e.g., oxidized neuroactive steroid or racemized or epimerized neuroactive steroid). For example, the sterilized buffered neuroactive steroid solutions or admixtures do not comprise more than 0.001, 0.002, 0.005, 0.01, 0.02, 0.05, 0.1, 0.2, 0.5, 1% w/w impurities. In some embodiments, the sterilized buffered neuroactive steroid solutions or admixtures have a pH of between about 3 and about 8 (e.g., between about 5 and about 7, between about 5.5 and about 6.5).

In some embodiments, the buffered neuroactive steroid solutions or admixtures are safe, well-tolerated, or non-irritating for human administration.

In some embodiments, the buffered neuroactive steroid as described herein is prepared as an emulsion suitable for parenteral administration. Such emulsions will contain a neuroactive steroid described herein in a suitable oil or mixture of oils, suitable emulsification ingredients, a suitable buffer, and other ingredients as needed to modify tonicity and to ensure the chemical and physical stability of the composition. As such they will be prepared by methods designed to ensure that they are sterile, and free of pyrogens, particulate matter, and other contaminants, and, where appropriate contain inhibitors of the growth of microorganisms. As such the buffered neuroactive steroid solutions will be essentially free of visible solid particles. In some embodiments, the buffered neuroactive steroid solutions described herein may be filtered. In some embodiments, the buffered neuroactive steroid solutions described herein can be sterilized (e.g., sterilized by filtration (e.g., filtered through 0.45 and 0.22 micron filters), by heat (e.g., steam sterilization at 121° C., or by irradiation, e.g., gamma irradiation). In some embodiments, the sterilized buffered neuroactive steroid emulsions maintain the required globule or droplet size to ensure safe and effective administration of the buffered neuroactive steroid emulsion. In some embodiments, the sterilized buffered neuroactive steroid emulsions do not comprise higher levels of impurities (e.g., oxidized neuroactive steroid or racemized or epimerized neuroactive steroid). For example, the sterilized buffered neuroactive steroid emulsion do not comprise more than 0.001, 0.002, 0.005, 0.01, 0.02, 0.05, 0.1, 0.2, 0.5, 1% w/w impurities. In some embodiments, the sterilized buffered neuroactive steroid emulsion has a pH of between about 3 and about 8 (e.g., between about 5 and about 7, between about 5.5 and about 6.5).

In some embodiments, the buffered neuroactive steroid is prepared as an oil solution suitable for injection. Such oil solutions will contain the neuroactive steroid in a suitable oil or mixture of oils and other ingredients as needed to ensure the chemical and physical stability of the composition. In some embodiments, the selection of oils and formulation excipients provide the desired release and sustained activity of the neuroactive steroid. As such they will be prepared by methods designed to ensure that they are sterile, and free of pyrogens, particulate matter, and other contaminants, and, where appropriate contain inhibitors of the growth of microorganisms. As such the buffered neuroactive steroid oil solution will be essentially free of visible solid particles. In some embodiments, the buffered neuroactive steroid oil solutions described herein may be filtered. In some embodiments, the buffered neuroactive steroid oil solution described herein can be sterilized (e.g., sterilized by filtration (e.g., filtered through 0.45 and 0.22 micron filters), by heat (dry heat sterilization >150° C.). In some embodiments, the sterilized buffered neuroactive steroid oil solution does not comprise higher levels of impurities (e.g., oxidized neuroactive steroid or racemized or epimerized neuroactive steroid). For example, the sterilized buffered neuroactive steroid oil solution does not comprise more than 0.001, 0.002, 0.005, 0.01, 0.02, 0.05, 0.1, 0.2, 0.5, 1% w/w impurities.

In some embodiments, the buffered neuroactive steroid solution or emulsion is lyophilized. Such lyophilized solution or emulsion may contain similar excipients as used for the neuroactive steroid solution described herein. In some embodiments the lyophilized buffered neuroactive solution or emulsion may contain additional components known to those skilled in art to enhance the lyophilization process such as but not limited to sugars, modified carbohydrate compounds, and solvents such as t-butyl alcohol. As such they will be prepared by methods designed to ensure that they are sterile, and free of pyrogens, particulate matter, and other contaminants, and, where appropriate contain inhibitors of the growth of microorganisms. As such the lyophilized buffered neuroactive steroid solution or emulsion will be essentially free of visible solid particles upon reconstitution. In some embodiments, the lyophilized buffered neuroactive steroid solution or emulsions described herein may be filtered prior to and after reconstitution. In some embodiments, the lyophilized buffered neuroactive steroid solution or emulsions described herein can be sterilized (e.g., sterilized by filtration (e.g., filtered through 0.45 and 0.22 micron filters) or by irradiation (e.g. gamma irradiation). In some embodiments, the lyophilized sterilized buffered neuroactive steroid solution or emulsions do not comprise more than 0.001, 0.002, 0.005, 0.01, 0.02, 0.05, 0.1, 0.2, 0.5, 1% w/w impurities (e.g., oxidized neuroactive steroid or racemized or epimerized neuroactive steroid). In some embodiments, the sterilized lyophilized buffered neuroactive steroid solution or emulsions have a pH of between about 3 and about 8 (e.g., between about 5 and about 7, between about 5.5 and about 6.5) after reconstitution.

### Admixture

The aqueous solutions described herein can be mixed with a diluent described herein to provide an “admixture”. Suitable diluents include sterile water for injection (“WFI”), saline, and dextrose. In some embodiments, an aqueous solution described herein is mixed with a diluent described herein in a ratio of 1:2 aqueous solution:diluent. In some embodiments, an aqueous solution described herein is mixed with a diluent described herein in a ratio of 1:9 aqueous solution:diluent.

In some embodiments, the admixture comprises about 1 to about 3 mg/mL neuroactive steroid. In some embodiments, the admixture comprises about 1.2 to about 2.5 mg/mL neuroactive steroid. In some embodiments, the admixture comprises about 1.4 to about 2.0 mg/mL neuroactive steroid. In some embodiments, the admixture comprises about 1.6 to about 1.7 mg/mL neuroactive steroid. In some embodiments, the admixture comprises about 1.67 mg/mL neuroactive steroid.

In some embodiments, the admixture comprises about 0.1 to about 1 mg/mL neuroactive steroid. In some embodiments, the admixture comprises about 0.25 to about 0.75 mg/mL neuroactive steroid. In some embodiments, the admixture comprises about 0.5 mg/mL neuroactive steroid.

In some embodiments, the admixture comprises about 1% to about 20% w/w cyclodextrin, e.g., sulfoalkylether-βcyclodextrin. In some embodiments, the admixture comprises about 2.5% to about 15% w/w cyclodextrin, e.g., sulfoalkylether-βcyclodextrin. In some embodiments, the admixture comprises about 5% to about 15% w/w cyclodextrin, e.g., sulfoalkylether-βcyclodextrin. In some embodiments, the admixture comprises about 5% to about 10% w/w cyclodextrin, e.g., sulfoalkylether-βcyclodextrin. In some embodiments, the admixture comprises about 8.3% w/w cyclodextrin, e.g., sulfoalkylether-β-cyclodextrin.

In some embodiments, the admixture comprises about 0.1% to about 10% w/w cyclodextrin, e.g., sulfoalkylether-βcyclodextrin. In some embodiments, the admixture comprises about 0.5% to about 7.5% w/w cyclodextrin, e.g., sulfoalkylether-βcyclodextrin. In some embodiments, the admixture comprises about 0.5% to about 5% w/w cyclodextrin, e.g., sulfoalkylether-βcyclodextrin. In some embodiments, the admixture comprises about 1% to about 5% w/w cyclodextrin, e.g., sulfoalkylether-βcyclodextrin. In some embodiments, the admixture comprises about 2.5% w/w cyclodextrin, e.g., sulfoalkylether-β-cyclodextrin.

In some embodiments, the admixture comprises about 1 to about 3 mg/mL neuroactive steroid and about 1% to about 20% w/w cyclodextrin, e.g., sulfoalkylether-βcyclodextrin. In some embodiments, the admixture comprises about 1.2 to about 2.5 mg/mL neuroactive steroid and about 2.5% to about 15% w/w cyclodextrin, e.g., sulfoalkylether-βcyclodextrin. In some embodiments, the admixture comprises about 1.4 to about 2.0 mg/mL neuroactive steroid and about 5% to about 15% w/w cyclodextrin, e.g., sulfoalkylether-βcyclodextrin. In some embodiments, the admixture comprises about 1.6 to about 1.7 mg/mL neuroactive steroid and about 5% to about 10% w/w cyclodextrin, e.g., sulfoalkylether-β-cyclodextrin. In some embodiments, the admixture comprises about 1.67 mg/mL neuroactive steroid and about 8.3% w/w cyclodextrin, e.g., sulfoalkylether-βcyclodextrin.

In some embodiments, the admixture comprises about 0.1 to about 1 mg/mL neuroactive steroid and about 0.1% to about 10% w/w cyclodextrin, e.g., sulfoalkylether-βcyclodextrin. In some embodiments, the admixture comprises about 0.25 to about 0.75 mg/mL neuroactive steroid and comprises about 0.5% to about 5% w/w cyclodextrin, e.g., sulfoalkylether-βcyclodextrin. In some embodiments, the admixture comprises about 0.5 mg/mL neuroactive steroid and about 2.5% w/w cyclodextrin, e.g., sulfoalkylether-βcyclodextrin.

In some embodiments, the admixture comprises a buffer described herein, e.g., a citrate buffer, phosphate buffer. In some embodiments, the buffer is present at about 1 to about 500 mM (e.g., about 1 to about 250 mM, about 1 to about 200 mM, about 1 to about 150 mM, about 1 to about 100 mM, about 1 to about 50 mM). In some embodiments, the buffer is at or near physiological pH. Preferably, the pH of the admixture is between about 3 to about 8 (e.g., between about 5 and about 7, between about 5.5 and about 6.5, between about 5.9 and about 6.1), or any specific value within said range. In some embodiments, the pH of the admixture is between about 5 to about 6.5, or any specific value within said range (e.g., 5.5, 5.6, 5.7, 5.8, 5.9, 6, 6.1, 6.2, 6.3, 6.4). In some embodiments, the pH of the admixture is about 6. In some embodiments, the buffer is citrate buffer and the pH is between about 3 to about 7.4. In some embodiments, the buffer is citrate buffer and the pH is between about 5.5 to about 6.2. In some embodiments, the buffer is phosphate buffer and the pH is between about 6.2 to 8.2, preferably about 7.4.

In some embodiments, the admixture comprises one part buffered neuroactive steroid solution (a buffered neuroactive steroid solution as described herein) per two parts diluent (e.g., WFI).

In some embodiments, the admixture comprises one part buffered neuroactive steroid solution (a buffered neuroactive steroid solution as described herein) per nine parts diluent (e.g., saline, WFI).

In some embodiments, the admixture is isotonic. In some embodiments, the admixture is hypotonic. In some embodiments, the tonicity of the admixture is adjusted, e.g., by tonicity enhancers, to provide solutions that are about 300 mOsm/L or less.

### Buffers

The aqueous neuroactive steroid solution or admixture described herein comprise a buffer (e.g., a buffer at a pH of between about 3 and about 8 (e.g., between about 5 and about 7, between about 5.5 and about 6.5, between about 5.9 and about 6.1). As used herein, the terms “buffer,” “buffer system,” or “buffering component” refers to a compound that, usually in combination with at least one other compound, provides a chemical system in solution that exhibits buffering capacity, that is, the capacity to neutralize, within limits, the pH lowering or raising effects of either strong acids or bases (alkali), respectively, with relatively little or no change in the original pH (e.g., the pH before being affected by, e.g., strong acid or base). For example, a buffer described herein maintains or controls the pH of a solution to a certain pH range. For example, “buffering capacity” can refer to the millimoles (mM) of strong acid or base (or respectively, hydrogen or hydroxide ions) required to change the pH by one unit when added to one liter (a standard unit) of the buffer solution. From this definition, it is apparent that the smaller the pH change in a solution caused by the addition of a specified quantity of acid or alkali, the greater the buffer capacity of the solution. See, for example, Remington: The Science and Practice of Pharmacy, Mack Publishing Co., Easton, Pa. (19^{th} Edition, 1995), Chapter 17, pages 225-227. The buffer capacity will depend on the kind and concentration of the buffer components.

According to some embodiments, the buffering components are present from 1 mM, 2 mM, 5 mM, 10 mM, 20 mM, 50 mM, 75 mM, 100 mM, 150 mM, 200 mM, 250 mM or more in solution.

Preferred buffers include 4-2-hydroxyethyl-1-piperazineethanesulfonic acid (HEPES), 2-{[tris(hydroxymethyl)methyl]amino}ethanesulfonic acid) (TES), 3-(N-morpholino)propanesulfonic acid (MOPS), piperazine-N,N′-bis(2-ethanesulfonic acid) (PIPES), dimethylarsinic acid (cacodylate), citrate (e.g., saline sodium citrate, potassium citrate, ammonium citrate), 2-(N-morpholino)ethanesulfonic acid (MES), phosphate (e.g., PBS, D-PBS), succinate (i.e., 2(R)-2-(methylamino)succinic acid), acetate, dimethylglutarate, maleate, imidazole, N-(2-Acetamido)-2-aminoethanesulfonic acid (ACES), N,N-bis(2-hydroxyethyl)-2-aminoethanesulfonic acid (BES), Bicine, Bis-Tris, Borate, N-cyclohexyl-3-aminopropanesulfonic acid (CAPS), Glycine, 3-[4-(2-Hydroxyethyl)-1-piperazinyl]propanesulfonic acid (HEPPS or EPPS), N-[Tris(hydroxymethyl)methyl]-3-aminopropanesulfonic acid, [(2-Hydroxy-1,1-bis(hydroxymethyl)ethyl)amino]-1-propanesulfonic acid (TAPS), Tricine, Tris, Tris Base, Tris Buffer, Tris-Glycine, Tris-HCl, collidine, veronal acetate, N-(2-Acetamido)iminodiacetic acid; N-(Carbamoylmethyl)iminodiacetic acid (ADA), β-Hydroxy-4-morpholinepropanesulfonic acid, 3-Morpholino-2-hydroxypropanesulfonic acid (MOPSO), cholamine chloride, 3-(N,N-Bis[2-hydroxyethyl]amino)-2-hydroxypropanesulfonic acid (DIPSO), acetamidoglycine, 3-{[1,3-Dihydroxy-2-(hydroxymethyl)-2-propanyl]amino}-2-hydroxy-1-propanesulfonic acid (TAPSO), Piperazine-N,N′-bis(2-hydroxypropanesulfonic acid) (POPSO), N-(2-Hydroxyethyl)piperazine-N′-(2-hydroxypropanesulfonic acid) (HEPPSO), N-cycloxhexyl-2-aminoethanesulfonic acid (CHES), 2-amino-methyl-1,3-proponediol (AMPd), and glycinamide.

In some embodiments, the buffer comprises a monoprotic acid. In some embodiments, the buffer comprises a polyprotic acid (e.g., citrate or phosphate). In some embodiments, the buffer is a solution of one or more substances (e.g., a salt of a weak acid and a weak base; a mixture of a weak acid and a salt of the weak acid with a strong base). In some embodiments, the buffer comprises a piperazine (e.g., PIPES, HEPES, POPSO, EPPS).

In some embodiments, the buffer comprises a non-metal complexing compound (e.g., MES, MOPS, PIPES).

In some embodiments, the buffer comprises a metal complexing compound (i.e., a metal chelating agent). In some embodiments, the metal chelating agent is citrate.

In some embodiments, the buffer is citrate buffer. In some embodiments, the buffer is phosphate buffer. In some embodiments, the buffer is histidine buffer.

In some embodiments, the buffer is present at a concentration of about 0.01, 0.05, 0.1, 0.5, 1, 5, 10, 20, 50, 100, 200, 250, 500 mM or more. In some embodiments, the buffer is present at a concentration of about 1 to about 500 mM, about 1 to about 300 mM, about 1 to about 200 mM, about 1 to about 100 mM, about 1 to about 50 mM, about 10 to about 500 mM, about 10 to about 300 mM, about 10 to about 200 mM, about 10 to about 100 mM, about 10 to about 50 mM.

In some embodiments, the buffer is present at a concentration of about 0.01 to about 10 mM, about 0.05 to about 5 mM, about 0.05 to about 5 mM, about 0.1 to about 5 mM, about 0.1 to about 3.5 mM.

In some embodiments, the pH of the aqueous solution is at or near physiological pH. Preferably, the pH of the aqueous solution is between about 3 to about 8 (e.g., between about 5 and about 7, between about 5.5 and about 6.5, between about 5.9 and about 6.1), or any specific value within said range. In some embodiments, the pH of the aqueous solution is between about 5 to about 6.5, or any specific value within said range (e.g., 5.5, 5.6, 5.7, 5.8, 5.9, 6, 6.1, 6.2, 6.3, 6.4). In some embodiments, the pH of the aqueous solution is about 6. The skilled artisan would recognize that the pH may be adjusted to a more optimal pH depending on the stability of the neuroactive steroids and sulfoalkylether-β-cyclodextrin included in the solution. The pH can be adjusted, for example, with hydrochloric, phosphoric acid or organic acids, such as citric acid, lactic acid, malic acid, tartaric acid, acetic acid, gluconic acid, succinic acid, and combinations thereof. In some embodiments, the pH is adjusted with base (e.g., 1 N sodium hydroxide) or acid (e.g., 1 N hydrochloric acid).

In some embodiments, the buffer is citrate buffer and the pH is between about 3 to about 8. In some embodiments, the buffer is citrate buffer and the pH is between about 3 to about 7.4. In some embodiments, the buffer is citrate buffer and the pH is between about 5.5 to about 6.2.

In some embodiments, the buffer is phosphate buffer and the pH is between about 3 to about 9. In some embodiments, the buffer is phosphate buffer and the pH is between about 6.2 to about 8.2. In some embodiments, the buffer is phosphate buffer and the pH is about 7.4.

### Neuroactive Steroids

The aqueous solutions or admixtures described herein comprise a neuroactive steroid described herein. Neuroactive steroids (or neurosteroids) are natural, synthetic, or semi-synthetic steroids that rapidly alter neuronal excitability through interaction with neurotransmitter-gated ion channels. Neuroactive steroids effect binding to membrane-bound receptors such as those for inhibitory and (or) excitatory neurotransmitters including GABA_{A}, NMDA, and sigma receptors.

The steroids that may be classified into functional groups according to chemical structure and physiological activity and include estrogenic hormones, progestational hormones, and androgenic hormones. Of particular interest are progestational hormones, referred to herein as “progestins” or “progestogens”, and their derivatives and bioactive metabolites. Members of this broad family include steroid hormones disclosed in Remington's Pharmaceutical Sciences, Gennaro et al., Mack Publishing Co. (18th ed. 1990), 990-993. As with all other classes of steroids, stereoisomerism is of fundamental importance with the sex hormones. As used herein, a variety of progestins (e.g., progesterone) and their derivatives, including both synthetic and natural products, can be used, as well as progestin metabolites such as progesterone.

The term “progesterone” as used herein refers to a member of the progestin family and includes a 21 carbon steroid hormone. Progesterone is also known as D4-pregnene-3,20-dione; Δ4-pregnene-3,20-dione; or pregn-4-ene-3,20-dione. As used herein a “synthetic progestin” is a molecule whose structure is related to that of progesterone, is synthetically derived, and retains the biological activity of progesterone.

Representative synthetic progestins include, but are not limited to, substitutions at the 17-position of the progesterone ring to introduce a hydroxyl, acetyl, hydroxyl acetyl, aliphatic, nitro, or heterocyclic group, modifications to produce 17α-OH esters (e.g., 17 α-hydroxyprogesterone caproate), as well as modifications that introduce 6-methyl, 6-ene, and 6-chloro substituents onto progesterone (e.g., medroxyprogesterone acetate, megestrol acetate, and chlomadinone acetate), and which retains the biological activity of progesterone. Such progestin derivatives include 5-dehydroprogesterone, 6-dehydro-retroprogesterone (dydrogesterone), allopregnanolone (allopregnan-3α, or 3β-ol-20-one), ethynodiol diacetate, hydroxyprogesterone caproate (pregn-4-ene-3,20-dione, 17-(1-oxohexy)oxy); levonorgestrel, norethindrone, norethindrone acetate (19-norpregn-4-en-20-yn-3-one, 17-(acetyloxy)-,(17α)-); norethynodrel, norgestrel, pregnenolone, ganaxolone (also referred to as CCD-1042 or INN), and megestrol acetate. In some embodiments, the neuroactive steroid is ganaxolone.

Useful progestins also can include allopregnone-3α or 3β, 20α or 20β-diol (see Merck Index 258-261); allopregnane-3β,21-diol-11,20-dione; allopregnane-3β,17α-diol-20-one; 3,20-allopregnanedione, allopregnane, 3β,11β,17α,20β,21-pentol; allopregnane-3β,17α,20β,21-tetrol; allopregnane-3α or 3β,11β,17α,21-tetrol-20-one, allopregnane-3β,17α or 20β-triol; allopregnane-3β,17α,21-triol-11,20-dione; allopregnane-3β, 11β, 21-triol-20-one; allopregnane-3β, 17α, 21-triol-20-one; allopregnane-3α or 3β-ol-20-one; pregnanediol; 3,20-pregnanedione; pregnan-3α-ol-20-one; 4-pregnene-20,21-diol-3,11-dione; 4-pregnene-11β, 17α, 20β, 21-tetrol-3-one; 4-pregnene-17α, 20β, 21-triol-3,11-dione; 4-pregnene-17α, 20β, 21-triol-3-one, and pregnenolone methyl ether. Further progestin derivatives include esters with non-toxic organic acids such as acetic acid, benzoic acid, maleic acid, malic acid, caproic acid, and citric acid and inorganic salts such as hydrochloride, sulfate, nitrate, bicarbonate and carbonate salts. Other suitable progestins include alphaxalone (also referred to as INN, alfaxolone, and alphaxolone), alphadolone (also referred to as alfadolone), hydroxydione, and minaxolone. In some embodiments, the neuroactive steroid is alphaxolone.

Additional suitable neuroactive steroids are disclosed in WIPO Publication Nos. WO2013/188792, WO 2013/056181, WO2015/010054, WO2014/169832, WO2014/169836, WO2014/169833, WO2014/169831, WO2015/027227, WO 2014/100228 and U.S. Pat. No. 5,232,917, U.S. Pat. No. 8,575,375 and U.S. Pat. No. 8,759,330, which are incorporated herein by reference for the neuroactive steroids described therein.

In particular embodiments, the steroids are one or more of a series of sedative-hypnotic 3 alpha-hydroxy ring A-reduced pregnane steroids that include the major metabolites of progesterone and deoxycorticosterone, 3 alpha-hydroxy-5 alpha-pregnan-20-one (allopregnanolone) and 3 alpha,21-dihydroxy-5 alpha-pregnan-20-one (allotetrahydroDOC), respectively. These 3 alpha-hydroxysteroids do not interact with classical intracellular steroid receptors but bind stereoselectively and with high affinity to receptors for the major inhibitory neurotransmitter in the brain, gamma-amino-butyric acid (GABA).

In certain embodiments, the neuroactive steroids is progesterone, pregnanolone, allopregnanolone, alphadalone, ganxolone, alphaxolone or other progesterone analogs. In a particular embodiment, the neuroactive steroid is allopregnanolone or a derivative thereof. In some embodiments, the neuroactive steroid is allopregnanolone. Exemplary derivatives include, but are not limited to, (20R)-17beta-(1-hydroxy-2,3-butadienyl)-5alpha-androstane-3alpha-ol (HBAO). Additional derivatives are described in WO 2012/127176.

In some embodiments, the neuroactive steroid is allopregnanolone. In some embodiments, the neuroactive steroid is ganaxolone. In some embodiments, the neuroactive steroid is alphaxolone.

The lipophilic nature of a neuroactive steroid (e.g., pregnanolone, allopregnanolone, alphadalone, ganxolone, or alphaxolone), can make it different to formulate for in vivo administration. As discussed above, the neuroactive steroid (e.g., pregnanolone, allopregnanolone, alphadalone, ganxolone, or alphaxolone), can be formulated with a host, such as a cyclodextrin to improve the solubility. Alternatively, or additionally, the neuroactive steroid (e.g., pregnanolone, allopregnanolone, alphadalone, ganxolone, or alphaxolone), can be modified in an attempt to improve the solubility. For example, polar groups can be introduced onto position 16α with the goal of increasing water solubility, brain accessibility, and potency of neuroactive steroids as described in Kasal et al., J. Med. Chem., 52(7), 2119-215 (2009).

### Cyclodextrins

The aqueous neuroactive steroid solution or admixture described herein comprise a cyclodextrin. The solubility of neuroactive steroids can be improved by cyclodextrins. Steroid-cyclodextrin complexes are known in the art. See, for example, U.S. Pat. No. 7,569,557 to Backensfeld, et al., and U.S. Patent Application Publication No. US 2006/0058262 to Zoppetti, et al.

Cyclodextrins are cyclic oligosaccharides containing or comprising six (α-cyclodextrin), seven (β-cyclodextrin), eight (γ-cyclodextrin), or more α-(1,4)-linked glucose residues. The hydroxyl groups of the cyclodextrins are oriented to the outside of the ring while the glucosidic oxygen and two rings of the non-exchangeable hydrogen atoms are directed towards the interior of the cavity.

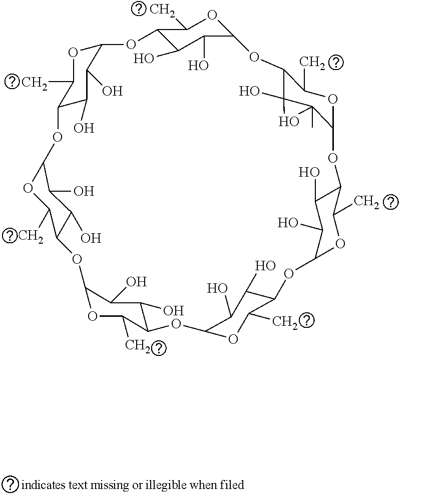 [SMILES: CC1OC2OC3C(C)OC(OC4C(C)OC(OC5C(C)OC(OC6C(C)OC(OC7C(C)OC(OC8C(C)OC(OC1C(O)C2O)C(O)C8O)C(O)C7O)C(O)C6O)C1(C)OC51O)C(O)C4O)C(O)C3O]

Neuroactive steroid-cyclodextrin complexes are preferably formed from a cyclodextrin selected from the group consisting of β-cyclodextrin, and derivatives thereof. The cyclodextrin may be chemically modified such that some or all of the primary or secondary hydroxyl groups of the macrocycle, or both, are functionalized with a pendant group. Suitable pendant groups include, but are not limited to, sulfinyl, sulfonyl, phosphate, acyl, and C₁-C₁₂ alkyl groups optionally substituted with one or more (e.g., 1, 2, 3, or 4) hydroxy, carboxy, carbonyl, acyl, oxy, oxo; or a combination thereof. Methods of modifying these alcohol residues are known in the art, and many cyclodextrin derivatives are commercially available, including sulfo butyl ether β-cyclodextrins available under the trade name CAPTISOL® from Ligand Pharmaceuticals (La Jolla, Calif.).

Preferred cyclodextrins include, but are not limited to, alkyl cyclodextrins, hydroxy alkyl cyclodextrins, such as hydroxy propyl β-cyclodextrin, carboxy alkyl cyclodextrins and sulfoalkyl ether cyclodextrins, such as sulfo butyl ether β-cyclodextrin.

In particular embodiments, the cyclodextrin is beta cyclodextrin having a plurality of charges (e.g., negative or positive) on the surface. In more particular embodiments, the cyclodextrin is a β-cyclodextrin containing or comprising a plurality of functional groups that are negatively charged at physiological pH. Examples of such functional groups include, but are not limited to, carboxylic acid (carboxylate) groups, sulfonate (RSO₃⁻), phosphonate groups, phosphinate groups, and amino acids that are negatively charged at physiological pH. The charged functional groups can be bound directly to the cyclodextrins or can be linked by a spacer, such as an alkylene chain. The number of carbon atoms in the alkylene chain can be varied, but is generally between about 1 and 10 carbons, preferably 1-6 carbons, more preferably 1-4 carbons. Highly sulfated cyclodextrins are described in U.S. Pat. No. 6,316,613.

In one embodiment, the cyclodextrins is a β-cyclodextrin functionalized with a plurality of sulfobutyl ether groups. Such a cyclodextrins is sold under the trade name CAPTISOL®.

CAPTISOL® is a polyanionic beta-cyclodextrin derivative with a sodium sulfonate salt separated from the lipophilic cavity by a butyl ether spacer group, or sulfobutylether (SBE). CAPTISOL® is not a single chemical species, but comprised of a multitude of polymeric structures of varying degrees of substitution and positional/regional isomers dictated and controlled to a uniform pattern by a patented manufacturing process consistently practiced and improved to control impurities.

CAPTISOL® contains six to seven sulfobutyl ether groups per cyclodextrin molecule. Because of the very low pKa of the sulfonic acid groups, CAPTISOL® carries multiple negative charges at physiologically compatible pH values. The four-carbon butyl chain coupled with repulsion of the end group negative charges allows for an “extension” of the cyclodextrin cavity. This often results in stronger binding to drug candidates than can be achieved using other modified cyclodextrins. It also provides a potential for ionic charge interactions between the cyclodextrin and a positively charged drug molecule. In addition, these derivatives impart exceptional solubility and parenteral safety to the molecule. Relative to beta-cyclodextrin, CAPTISOL® provides higher interaction characteristics and superior water solubility in excess of 100 grams/100 ml, a 50-fold improvement.

Preferably, the cyclodextrin is present in an amount of from about 0.1% to about 40% w/w of the overall solution (e.g., buffered neuroactive steroid solution), preferably from about 5% to about 40% w/w, more preferably about 10% to about 40% w/w, most preferably about 10% to about 35% w/w. In certain embodiments, the concentration of the cyclodextrins is from about 15% to about 35% w/w, preferably from about 20% to about 35% w/w, more preferably about 20% to about 30% w/w. In certain embodiments, the concentration of the cyclodextrins is about 25% w/w.

In one embodiment, the formulation contains about 1 to about 2, preferably about 1.5 mg neuroactive steroid (e.g., pregnanolone, allopregnanolone, alphadalone, ganaxolone, alphaxolone) per ml of cyclodextrin, e.g., CAPTISOL®. In some embodiments, the cyclodextrin, e.g., sulfoalkylether-β-cyclodextrin, is present in the aqueous solution described herein at 0.1, 0.2, 0.3, 0.5, 0.7, 1, 1.2, 1.5, 1.8, 2, 2.5, 3, 4, 5, 6, 7, 8, 10, 11, 12 mg/mL or more.

In some embodiments, the cyclodextrin, e.g., sulfoalkylether-βcyclodextrin, is present in the aqueous solution described herein at 1, 2, 3, 5, 7, 10, 12, 20, 25, 30, 40% w/w or more.

In some embodiments, the cyclodextrin, e.g., sulfoalkylether-βcyclodextrin, is present in the aqueous solution described herein at least 0.1, 0.2, 0.3, 0.5, 0.7, 1, 1.2, 1.5, 2, 3, 4, 5, 6, 7, 8, 10 mg/mL or more.

In some embodiments, the molar ratio of neuroactive steroid to cyclodextrin, e.g., sulfoalkylether-βcyclodextrin is about 0.1, 0.05, 0.03, 0.02, 0.01, 0.008, 0.005 or less.

### Tonicity Enhancers

The aqueous neuroactive steroid solution or admixture described herein may further comprise a tonicity enhancer. Tonicity is the effective osmotic pressure equivalent, or the relative concentration of solutions that determine the direction and extent of diffusion. Tonicity may be adjusted if needed typically by tonicity enhancing agents. Such agents may, for example be of ionic and/or non-ionic type. Examples of ionic tonicity enhancers are alkali metal or earth metal halides, such as, for example, CaCl₂, KBr, KCl, LiCl, NaI, NaBr or NaCl, Na₂SO₄, or boric acid. Non-ionic tonicity enhancing agents are, for example, urea, glycerol, sorbital, mannitol, propylene glycol, or dextrose. The aqueous solutions described are typically adjusted with tonicity agents to be isotonic (e.g., about 270 to about 300 mOsm/L, about 275 to about 295 mOsm/L). In some embodiments, the aqueous solutions described are adjusted with tonicity agents to an osmolarlity of ranging from about 150 to about 320 mOsm/L (e.g., about 200 to about 300 mOsm/L). In some embodiments, the aqueous solutions are less than about 320 mOsm/L (e.g., less than about 300, 290, 280, 270, 260, 250 mOsm/L).

In some embodiments, the aqueous solutions described are hypertonic. For example, the aqueous solutions may be hypertonic (e.g., about 900 to about 1000 mOsm/L). In some embodiments, the aqueous solutions are diluted with Water For Injection (“WFI”, e.g., highly purified water free of any added components; sterile, nonpyrogenic, solute-free preparation of distilled water for injection), e.g., to provide an isotonic or hypotonic solution. In some embodiments, the admixture is diluted with a solution of NaCl (e.g., saline).

### Preservatives

The aqueous neuroactive steroid solution or admixture described herein may include preservatives. Exemplary preservatives include antimicrobial agents (e.g., tissue plasminogen activator, sargramostim, interleukins, phenol, benzyl alcohol, meta-cresol, parabens (methyl, propyl, butyl), benzalkonium chloride, chlorobutanol, thimerosal, phenylmercuric salts (acetate, borate, nitrate)), benzalkonium chloride, benzethonium chloride, chlorobutanol, myristyl gamma-picolinium chloride, 2-phenoxyethanol, thiomerosal, methylparaben, propylparaben, butylparaben, ethylenediamine, formaldehyde.

The aqueous neuroactive steroid solution or admixture described herein may include antioxidants. Exemplary antioxidants include sodium bisulfite, sulfurous acid salts, ascorbic acid and its salts, acetylcysteine, monothioglyercol), EDTA, cryoprotectants and lyoprotectants (e.g., sugars (e.g., sucrose, trehalose), amino acids (e.g., glycine, lysine), polymers (e.g., liquid polyethylene glycol or dextran), polyols (e.g., mannitol, sorbitol)

### Sterilization

The aqueous neuroactive steroid solution or admixture described herein may require sterilization, e.g., before administration. The compositions described herein provide stability (e.g., chemical stability, physical stability) in the presence of sterilization processes. In some embodiments, the buffered neuroactive steroid solution or admixture is sterile. In some embodiments, the aqueous neuroactive steroid solution or admixture is sterilized through aseptic processing (e.g., aseptic fill, aseptic filtration). In some embodiments, the aqueous neuroactive steroid solution or admixture is sterilized through terminal sterilization (e.g. heat (such as dry heat or steam autoclave) or irradiation (such as gamma irradiation). The compositions described herein (e.g., compositions comprising a buffer as described herein) provide stability (e.g., chemical stability, physical stability) in the presence of terminal sterilization (e.g., at temperature cycles of from about 120° C. to about 124° C., e.g., 121° C.) or irradiation.

### Mixing

The aqueous neuroactive steroid solution or admixture described herein may require mixing, e.g., to provide homogeneous solutions or admixtures. In some embodiments, the manufacture of the buffered neuroactive steroid solution or emulsion requires vigorous, high intensity, high shear mixing (agitation). The agitation may be supplied with or without heating. In some embodiments, heating the mixture during agitation may facilitate the mixing efficiency and reduce the time required for dissolution or emulsification. The amount of heating (mixture temperature) applied is dependent on the system being mixed; but may be limited by the equipment operation and physical and chemical stability of the mixture. In some embodiments a temperature of about 40 C has been found useful to facilitate preparation of the product.

Agitation can be supplied by devices such as high shear impeller mixers, rotor stator mixers, homgenizers, ultrasonic devices, or microfluidizers. The vigorous, high intensity, high shear agitation or mixing is used to mix and blend two mutually non-soluble liquids or to facilitate the dissolution of solid particles into a vehicle to make the same or uniform throughout. High shear mixers function to induce fluid travel with a different velocity relative to the fluid in an adjacent area. The dissolution or emulsification may be achieved by turning one of the product phases into a state consisting of extremely small particles distributed uniformly throughout the other liquid.

Mixing with high shear impellers may provide sufficient agitation for dissolution of some embodiments of the neuroactive steroid solution or emulsification. However in some embodiments, the duration of mixing may be too long for practical manufacturing cycles. Agitation supplied by rotor stator mixers, homgenizers, ultrasonic devices, or microfluidizers may speed and facilitate dissolution to make a practical manufacturing cycle time. In some embodiments, heating the mixture during agitation may facilitate the mixing efficiency and reduce the time required for dissolution or emulsification. The amount of heating (mixture temperature) applied is dependent on the system being mixed; but may be limited by the equipment operation and physical and chemical stability of the mixture. In some embodiments a temperature of about 40° C. facilitates preparation of the product.

High-shear mixing devices such as rotor stator mixers may provide sufficient agitation for dissolution of some embodiments of the neuroactive steroid solution or emulsification. High rotor/stators use a rotating impeller or high-speed rotor typically powered by an electric motor. The rotor spins at very high speed (e.g. 2,000 to 18,000 RPM) in the mixture within a stationary ring (stator) to create flow and shear. Suction is created from the high-speed rotation of the rotor blades within the stator drawing the mixture into the center of the rotor/stator assembly. The high-speed centrifugal force drives the mixture towards the periphery of the rotor toward the stator where it is subjected to a milling action due to the restricted clearance between the rotor and the stator. The mixture is the forced by intense hydraulic shear, at high velocity, out through the perforations in the stator in into the mixing vessel. The effect of the horizontal (radial) expulsion and suction of the mixture into the rotor/stator, sets up a circulation pattern within the mixing vessel. The design of rotor and the design of the stator vary with the types and designs of the equipment; and one skilled in the art may find that numerous combinations of rotors and stator designs may function acceptably. The size of the rotor/stator assembly will be sized depending on the batch size and the desired duration of processing. The location of rotor/stator assembly will vary depending on the equipment design, but some embodiments may use a rotor/stator assembly mounted on or near the bottom of the mixing vessel. A top mounted rotor/stator that designed to be immersed in the mixture may be used. A rotor/stator assembly mounted external to the mixing vessel where the mixture is introduced and may be caused to pass through or be re-circulated through the rotor/stator head. The desired speed of the rotor within the stator is typically variable, and may be set to provide desired flow and high shear mixing within practical manufacturing cycles. Those skilled in the art will recognize that the tip speed of the rotor can be used to facilitate the scale-up of the size of the rotor/stator assembly as batch size is increased. In some embodiments, heating the mixture during agitation may facilitate the high shear mixing efficiency and reduce the time required for dissolution or emulsification. The amount of heating (mixture temperature) applied is dependent on the system being mixed; but may be limited by the equipment operation and physical and chemical stability of the mixture. In some embodiments a temperature of about 40° C. has been found useful to facilitate preparation of the product (e.g., an aqueous solution or admixture as described herein).

High shear mixing devices such as homogenizers may provide sufficient agitation for dissolution of some embodiments of the neuroactive steroid solution or emulsification. Homogenizers provide high shear as they function to pump the mixture at high pressure (e.g. 1000-5000 psi) into a small chamber that is comprised of a valve seat, an impact ring and the valve. The mixture flows at high pressure through the region between the valve and valve seat at high velocity with and under a rapid pressure drop. The rapid pressure drop disrupts the mixture by cavitation and the shock occurring when the cavitation bubble collapses. The mixture next strikes the impact ring causing additional disruption and shear within the mixture. The mixture is discharged into the bulk solution. Different valve assemblies, relative location of the emulsifier to the product batch, multiple valve assemblies, and equipment with a wide range of capacities can be used. In some embodiments, heating the mixture during agitation may facilitate the high shear mixing efficiency and reduce the time required for dissolution or emulsification. The amount of heating or temperature control of the mixing process (mixture temperature) applied is dependent on the system being mixed; but may be limited by the equipment operation and physical and chemical stability of the mixture. In some embodiments a temperature of about 40° C. has been found useful to facilitate preparation of the product (e.g., an aqueous solution or admixture as described herein).

High shear mixing devices such as microfluidizers may provide sufficient agitation for dissolution of some embodiments of the neuroactive steroid solution or emulsification. The high shear mixing from microfluidizers results is caused by pumping the mixture at extremely high velocity at high pressure (e.g. 2,000 to 40,000 psi) through small channels into an interaction chamber. In the interaction chamber the mixture is subjected to high shear, turbulence, impact, and cavitation. All of these forces can facilitate the high shear mixing efficiency and reduce the time required for dissolution or emulsification. Different interaction chamber assemblies, relative location of the microfluidizer to the product batch, and equipment with a wide range of capacities can be used. The amount of heating or temperature control of the mixing process (mixture temperature) applied is dependent on the system being mixed; but may be limited by the equipment operation and physical and chemical stability of the mixture. In some embodiments a temperature of about 40 C has been found useful to facilitate preparation of the product.

High shear mixing devices that use ultrasonic energy may provide sufficient agitation for dissolution of some embodiments of the neuroactive steroid solution or emulsification. The high shear mixing from ultrasonic energy results is caused by cavitation and rapid collapse of the small bubbles formed by the cavitation. These forces can facilitate the high shear mixing efficiency and reduce the time required for dissolution or emulsification. Different sonication assemblies, relative location of the sonication assembly to the product batch, and equipment with a wide range of capacities can be used. The amount of heating or temperature control of the mixing process (mixture temperature) applied is dependent on the system being mixed; but may be limited by the equipment operation and physical and chemical stability of the mixture. In some embodiments a temperature of about 40° C. has been found useful to facilitate preparation of the product.

### Containers

Also described herein are containers that include an aqueous solution or admixture described herein. Examples of containers include bags (e.g., plastic or polymer bags such as PVC), vials (e.g., a glass vial), bottles, or syringes. In an embodiment, the container is configured to deliver the solution or admixture parenterally (e.g., i.m. or i.v.).

In some embodiments, the product intended for injection is packed in a suitably sized hermetically sealed glass container. In some embodiments the product is intended to be diluted prior to infusion, and is packaged in a pharmaceutical vial or bottle (e.g. suitably sized, suitable glass or plastic vial or bottle). In some embodiments the product may prepared to be ready for injection and may be packaged in a prefilled syringe or other syringe device (e.g. suitably sized, suitable glass or plastic package) or large volume container (e.g. suitably sized, suitable glass or plastic container) intended to be used for infusion. In some embodiments, the product is provided in a container that does not leach (e.g., does not introduce (or allow growth of) contamination or impurities in the solution.

### Neurodegenerative Diseases and Disorders

The solutions or admixtures described herein can be used in a method described herein, for example in the treatment of a disorder described herein such as a neurodegenerative disease.

The term “neurodegenerative disease” includes diseases and disorders that are associated with the progressive loss of structure or function of neurons, or death of neurons. Neurodegenerative diseases and disorders include, but are not limited to, Alzheimer's disease (including the associated symptoms of mild, moderate, or severe cognitive impairment); amyotrophic lateral sclerosis (ALS); anoxic and ischemic injuries; ataxia and convulsion (including for the treatment and prevention and prevention of seizures that are caused by schizoaffective disorder or by drugs used to treat schizophrenia); benign forgetfulness; brain edema; cerebellar ataxia including McLeod neuroacanthocytosis syndrome (MLS); closed head injury; coma; contusive injuries (e.g., spinal cord injury and head injury); dementias including multi-infarct dementia and senile dementia; disturbances of consciousness; Down syndrome; drug-induced or medication-induced Parkinsonism (such as neuroleptic-induced acute akathisia, acute dystonia, Parkinsonism, or tardive dyskinesia, neuroleptic malignant syndrome, or medication-induced postural tremor); epilepsy; fragile X syndrome; Gilles de la Tourette's syndrome; head trauma; hearing impairment and loss; Huntington's disease; Lennox syndrome; levodopa-induced dyskinesia; mental retardation; movement disorders including akinesias and akinetic (rigid) syndromes (including basal ganglia calcification, corticobasal degeneration, multiple system atrophy, Parkinsonism-ALS dementia complex, Parkinson's disease, postencephalitic parkinsonism, and progressively supranuclear palsy); muscular spasms and disorders associated with muscular spasticity or weakness including chorea (such as benign hereditary chorea, drug-induced chorea, hemiballism, Huntington's disease, neuroacanthocytosis, Sydenham's chorea, and symptomatic chorea), dyskinesia (including tics such as complex tics, simple tics, and symptomatic tics), myoclonus (including generalized myoclonus and focal cyloclonus), tremor (such as rest tremor, postural tremor, and intention tremor) and dystonia (including axial dystonia, dystonic writer's cramp, hemiplegic dystonia, paroxysmal dystonia, and focal dystonia such as blepharospasm, oromandibular dystonia, and spasmodic dysphonia and torticollis); neuronal damage including ocular damage, retinopathy or macular degeneration of the eye; neurotoxic injury which follows cerebral stroke, thromboembolic stroke, hemorrhagic stroke, cerebral ischemia, cerebral vasospasm, hypoglycemia, amnesia, hypoxia, anoxia, perinatal asphyxia and cardiac arrest; Parkinson's disease; seizure; status epilecticus; stroke; tinnitus; tubular sclerosis, and viral infection induced neurodegeneration (e.g., caused by acquired immunodeficiency syndrome (AIDS) and encephalopathies). Neurodegenerative diseases also include, but are not limited to, neurotoxic injury which follows cerebral stroke, thromboembolic stroke, hemorrhagic stroke, cerebral ischemia, cerebral vasospasm, hypoglycemia, amnesia, hypoxia, anoxia, perinatal asphyxia and cardiac arrest. Methods of treating or preventing a neurodegenerative disease also include treating or preventing loss of neuronal function characteristic of neurodegenerative disorder.

### Mood Disorders

The solutions or admixtures described herein can be used in a method described herein, for example in the treatment of a disorder described herein such as a mood disorder.

Clinical depression is also known as major depression, major depressive disorder (MDD), severe depression, unipolar depression, unipolar disorder, and recurrent depression, and refers to a mental disorder characterized by pervasive and persistent low mood that is accompanied by low self-esteem and loss of interest or pleasure in normally enjoyable activities. Some people with clinical depression have trouble sleeping, lose weight, and generally feel agitated and irritable. Clinical depression affects how an individual feels, thinks, and behaves and may lead to a variety of emotional and physical problems. Individuals with clinical depression may have trouble doing day-to-day activities and make an individual feel as if life is not worth living.

Postnatal depression (PND) is also referred to as postpartum depression (PPD), and refers to a type of clinical depression that affects women after childbirth. Symptoms can include sadness, fatigue, changes in sleeping and eating habits, reduced sexual desire, crying episodes, anxiety, and irritability. In some embodiments, the PND is a treatment-resistant depression (e.g., a treatment-resistant depression as described herein). In some embodiments, the PND is refractory depression (e.g., a refractory depression as described herein).

Atypical depression (AD) is characterized by mood reactivity (e.g., paradoxical anhedonia) and positivity, significant weight gain or increased appetite. Patients suffering from AD also may have excessive sleep or somnolence (hypersomnia), a sensation of limb heaviness, and significant social impairment as a consequence of hypersensitivity to perceived interpersonal rejection.

Melancholic depression is characterized by loss of pleasure (anhedonia) in most or all activities, failures to react to pleasurable stimuli, depressed mood more pronounced than that of grief or loss, excessive weight loss, or excessive guilt.

Psychotic major depression (PMD) or psychotic depression refers to a major depressive episode, in particular of melancholic nature, where the individual experiences psychotic symptoms such as delusions and hallucinations.

Catatonic depression refers to major depression involving disturbances of motor behavior and other symptoms. An individual may become mute and stuporose, and either is immobile or exhibits purposeless or bizarre movements.

Seasonal affective disorder (SAD) refers to a type of seasonal depression wherein an individual has seasonal patterns of depressive episodes coming on in the fall or winter.

Dysthymia refers to a condition related to unipolar depression, where the same physical and cognitive problems are evident. They are not as severe and tend to last longer (e.g., at least 2 years).

Double depression refers to fairly depressed mood (dysthymia) that lasts for at least 2 years and is punctuated by periods of major depression.

Depressive Personality Disorder (DPD) refers to a personality disorder with depressive features.

Recurrent Brief Depression (RBD) refers to a condition in which individuals have depressive episodes about once per month, each episode lasting 2 weeks or less and typically less than 2-3 days.

Minor depressive disorder or minor depression refers to a depression in which at least 2 symptoms are present for 2 weeks.

Bipolar disorder or manic depressive disorder causes extreme mood swings that include emotional highs (mania or hypomania) and lows (depression). During periods of mania the individual may feel or act abnormally happy, energetic, or irritable. They often make poorly thought out decisions with little regard to the consequences. The need for sleep is usually reduced. During periods of depression there may be crying, poor eye contact with others, and a negative outlook on life. The risk of suicide among those with the disorder is high at greater than 6% over 20 years, while self harm occurs in 30-40%. Other mental health issues such as anxiety disorder and substance use disorder are commonly associated with bipolar disorder.

Depression caused by chronic medical conditions refers to depression caused by chronic medical conditions such as cancer or chronic pain, chemotherapy, chronic stress.

Treatment-resistant depression refers to a condition where the individuals have been treated for depression, but the symptoms do not improve. For example, antidepressants or physchological counseling (psychotherapy) do not ease depression symptoms for individuals with treatment-resistant depression. In some cases, individuals with treatment-resistant depression improve symptoms, but come back. Refractory depression occurs in patients suffering from depression who are resistant to standard pharmacological treatments, including tricyclic antidepressants, MAOIs, SSRIs, and double and triple uptake inhibitors and/or anxiolytic drugs, as well as non-pharmacological treatments (e.g., psychotherapy, electroconvulsive therapy, vagus nerve stimulation and/or transcranial magnetic stimulation).

Suicidality, suicidal ideation, suicidal behavior refers to the tendency of an individual to commit suicide. Suicidal ideation concerns thoughts about or an unusual preoccupation with suicide. The range of suicidal ideation varies greatly, from e.g., fleeting thoughts to extensive thoughts, detailed planning, role playing, incomplete attempts. Symptoms include talking about suicide, getting the means to commit suicide, withdrawing from social contact, being preoccupied with death, feeling trapped or hopeless about a situation, increasing use of alcohol or drugs, doing risky or self-destructive things, saying goodbye to people as if they won't be seen again.

Premenstrual dysphoric disorder (PMDD) refers to a severe, at times disabling extension of premenstrual syndrome (PMS). PMDD causes extreme modd shifts with symptoms that typically begin seven to ten days before a female's period starts and continues for the first few days of a female's period. Symptoms include sadness or hopelessness, anxiety or tension, extreme moodiness, and marked irritability or anger.

Symptoms of depression include persistent anxious or sad feelings, feelings of helplessness, hopelessness, pessimism, worthlessness, low energy, restlessness, irritability, fatigue, loss of interest in pleasurable activities or hobbies, absence of positive thoughts or plans, excessive sleeping, overeating, appetite loss, insomnia, self-harm, thoughts of suicide, and suicide attempts. The presence, severity, frequency, and duration of symptoms may vary on a case to case basis. Symptoms of depression, and relief of the same, may be ascertained by a physician or psychologist (e.g., by a mental state examination).

### Anxiety Disorders

The solutions or admixtures described herein can be used in a method described herein, for example in the treatment of a disorder described herein such as an anxiety disorder.

Anxiety disorder is a blanket term covering several different forms of abnormal and pathological fear and anxiety. Current psychiatric diagnostic criteria recognize a wide variety of anxiety disorders.

Generalized anxiety disorder is a common chronic disorder characterized by long-lasting anxiety that is not focused on any one object or situation. Those suffering from generalized anxiety experience non-specific persistent fear and worry and become overly concerned with everyday matters. Generalized anxiety disorder is the most common anxiety disorder to affect older adults.

In panic disorder, a person suffers from brief attacks of intense terror and apprehension, often marked by trembling, shaking, confusion, dizziness, nausea, difficulty breathing. These panic attacks, defined by the APA as fear or discomfort that abruptly arises and peaks in less than ten minutes, can last for several hours and can be triggered by stress, fear, or even exercise; although the specific cause is not always apparent. In addition to recurrent unexpected panic attacks, a diagnosis of panic disorder also requires that said attacks have chronic consequences: either worry over the attacks' potential implications, persistent fear of future attacks, or significant changes in behavior related to the attacks. Accordingly, those suffering from panic disorder experience symptoms even outside of specific panic episodes. Often, normal changes in heartbeat are noticed by a panic sufferer, leading them to think something is wrong with their heart or they are about to have another panic attack. In some cases, a heightened awareness (hypervigilance) of body functioning occurs during panic attacks, wherein any perceived physiological change is interpreted as a possible life threatening illness (i.e. extreme hypochondriasis).

Obsessive compulsive disorder is a type of anxiety disorder primarily characterized by repetitive obsessions (distressing, persistent, and intrusive thoughts or images) and compulsions (urges to perform specific acts or rituals). The OCD thought pattern may be likened to superstitions insofar as it involves a belief in a causative relationship where, in reality, one does not exist. Often the process is entirely illogical; for example, the compulsion of walking in a certain pattern may be employed to alleviate the obsession of impending harm. And in many cases, the compulsion is entirely inexplicable, simply an urge to complete a ritual triggered by nervousness. In a minority of cases, sufferers of OCD may only experience obsessions, with no overt compulsions; a much smaller number of sufferers experience only compulsions.

The single largest category of anxiety disorders is that of phobia, which includes all cases in which fear and anxiety is triggered by a specific stimulus or situation. Sufferers typically anticipate terrifying consequences from encountering the object of their fear, which can be anything from an animal to a location to a bodily fluid.

Post-traumatic stress disorder or PTSD is an anxiety disorder which results from a traumatic experience. Post-traumatic stress can result from an extreme situation, such as combat, rape, hostage situations, or even serious accident. It can also result from long term (chronic) exposure to a severe stressor, for example soldiers who endure individual battles but cannot cope with continuous combat. Common symptoms include flashbacks, avoidant behaviors, and depression.

### Eating Disorders

The solutions or admixtures described herein can be used in a method described herein, for example in the treatment of a disorder described herein such as an eating disorder. Eating disorders feature disturbances in eating behavior and weight regulation, and are associated with a wide range of adverse psychological, physical, and social consequences. An individual with an eating disorder may start out just eating smaller or larger amounts of food, but at some point, their urge to eat less or more spirals out of control. Eating disorders may be characterized by severe distress or concern about body weight or shape, or extreme efforts to manage weight or food intake. Eating disorders include anorexia nervosa, bulimia nervosa, binge-eating disorder, cachexia, and their variants.

Individuals with anorexia nervosa typically see themselves as overweight, even when they are underweight. Individuals with anorexia nervosa can become obsessed with eating, food, and weight control. Individuals with anorexia nervosa typically weigh themselves repeatedly, portion food carefully, and eat very small quantities of only certain foods. Individuals with anorexia nervosa may engage in binge eating, followed by extreme dieting, excessive exercise, self-induced vomiting, or misuse of laxatives, diuretics, or enemas. Symptoms include extremely low body weight, severe food restriction, relentless pursuit of thinness and unwillingness to maintain a normal or healthy weight, intense fear of gaining weight, distorted body image and self-esteem that is heavily influenced by perceptions of body weight and shape, or a denial of the seriousness of low body weight, lack of menstruation among girls and women. Other symptoms include the thinning of the bones, brittle hair and nails, dry and yellowish skin, growth of fine hair all over the body, mild anemia, muscle wasting, and weakness, severe constipation, low blood pressure or slowed breathing and pulse, damage to the structure and function of the heart, brain damage, multi-organ failure, drop in internal body temperature, lethargy, sluggishness, and infertility.

Individuals with bulimia nervosa have recurrent and frequent episodes of eating unusually large amounts of food and feel a lack of control over these episodes. This binge eating is followed by behavior that compensates for the overeating such as forced vomiting, excessive use of laxatives or diuretics, fasting, excessive exercise, or a combination of these behaviors.

Unlike anorexia nervosa, people with bulimia nervosa usually maintain what is considered a healthy or normal weight, while some are slightly overweight. But like people with anorexia nervosa, they typically fear gaining weight, want desperately to lose weight, and are unhappy with their body size and shape. Usually, bulimic behavior is done secretly because it is often accompanied by feelings of disgust or shame. The binge eating and purging cycle can happen anywhere from several times a week to many times a day. Other symptoms include chronically inflamed and sore throat, swollen salivary glands in the neck and jaw area, worn tooth enamel, and increasingly sensitive and decaying teeth as a result of exposure to stomach acid, acid reflux disorder and other gastrointestinal problems, intestinal distress and irritation from laxative abuse, severe dehydration from purging of fluids, electrolyte imbalance (that can lead to a heart attack or stroke).

Individuals with binge-eating disorder lose control over their eating. Unlike bulimia nervosa, periods of binge eating are not followed by compensatory behaviors like purging, excessive exercise, or fasting. Individuals with binge-eating disorder often are overweight or obese. Obese individuals with binge-eating disorder are at higher risk for developing cardiovascular disease and high blood pressure. They also experience guilt, shame, and distress about their binge eating, which can lead to more binge eating.

Cachexia is also known as “wasting disorder,” and is an eating-related issue experienced by many cancer patients. Individuals with cachexia may continue to eat normally, but their body may refuse to utilize the vitamins and nutrients that it is ingesting, or they will lose their appetite and stop eating. When an individual experiences loss of appetite and stops eating, they can be considered to have developed anorexia nervosa.

### Epilepsy

The solutions or admixtures described herein can be used in a method described herein, for example in the treatment of a disorder described herein such as epilepsy, status epilepticus, or seizure, for example as described in WO2013/112605 and WO/2014/031792, the contents of which are incorporated herein in their entirety.

Epilepsy is a brain disorder characterized by repeated seizures over time. Types of epilepsy can include, but are not limited to generalized epilepsy, e.g., childhood absence epilepsy, juvenile nyoclonic epilepsy, epilepsy with grand-mal seizures on awakening, West syndrome, Lennox-Gastaut syndrome, partial epilepsy, e.g., temporal lobe epilepsy, frontal lobe epilepsy, benign focal epilepsy of childhood.

Status epilepticus (SE)

Status epilepticus (SE) can include, e.g., convulsive status epilepticus, e.g., early status epilepticus, established status epilepticus, refractory status epilepticus, super-refractory status epilepticus; non-convulsive status epilepticus, e.g., generalized status epilepticus, complex partial status epilepticus; generalized periodic epileptiform discharges; and periodic lateralized epileptiform discharges. Convulsive status epilepticus is characterized by the presence of convulsive status epileptic seizures, and can include early status epilepticus, established status epilepticus, refractory status epilepticus, super-refractory status epilepticus. Early status epilepticus is treated with a first line therapy. Established status epilepticus is characterized by status epileptic seizures which persist despite treatment with a first line therapy, and a second line therapy is administered. Refractory status epilepticus is characterized by status epileptic seizures which persist despite treatment with a first line and a second line therapy, and a general anesthetic is generally administered. Super refractory status epilepticus is characterized by status epileptic seizures which persist despite treatment with a first line therapy, a second line therapy, and a general anesthetic for 24 hours or more.

Non-convulsive status epilepticus can include, e.g., focal non-convulsive status epilepticus, e.g., complex partial non-convulsive status epilepticus, simple partial non-convulsive status epilepticus, subtle non-convulsive status epilepticus; generalized non-convulsive status epilepticus, e.g., late onset absence non-convulsive status epilepticus, atypical absence non-convulsive status epilepticus, or typical absence non-convulsive status epilepticus.

Compositions described herein can also be administered as a prophylactic to a subject having a CNS disorder e.g., a traumatic brain injury, status epilepticus, e.g., convulsive status epilepticus, e.g., early status epilepticus, established status epilepticus, refractory status epilepticus, super-refractory status epilepticus; non-convulsive status epilepticus, e.g., generalized status epilepticus, complex partial status epilepticus; generalized periodic epileptiform discharges; and periodic lateralized epileptiform discharges; prior to the onset of a seizure.

### Seizure

A seizure is the physical findings or changes in behavior that occur after an episode of abnormal electrical activity in the brain. The term “seizure” is often used interchangeably with “convulsion.” Convulsions are when a person's body shakes rapidly and uncontrollably. During convulsions, the person's muscles contract and relax repeatedly.

Based on the type of behavior and brain activity, seizures are divided into two broad categories: generalized and partial (also called local or focal). Classifying the type of seizure helps doctors diagnose whether or not a patient has epilepsy.

Generalized seizures are produced by electrical impulses from throughout the entire brain, whereas partial seizures are produced (at least initially) by electrical impulses in a relatively small part of the brain. The part of the brain generating the seizures is sometimes called the focus.

There are six types of generalized seizures. The most common and dramatic, and therefore the most well known, is the generalized convulsion, also called the grand-mal seizure. In this type of seizure, the patient loses consciousness and usually collapses. The loss of consciousness is followed by generalized body stiffening (called the “tonic” phase of the seizure) for 30 to 60 seconds, then by violent jerking (the “clonic” phase) for 30 to 60 seconds, after which the patient goes into a deep sleep (the “postictal” or after-seizure phase). During grand-mal seizures, injuries and accidents may occur, such as tongue biting and urinary incontinence.

Absence seizures cause a short loss of consciousness (just a few seconds) with few or no symptoms. The patient, most often a child, typically interrupts an activity and stares blankly. These seizures begin and end abruptly and may occur several times a day. Patients are usually not aware that they are having a seizure, except that they may be aware of “losing time.”

Myoclonic seizures consist of sporadic jerks, usually on both sides of the body. Patients sometimes describe the jerks as brief electrical shocks. When violent, these seizures may result in dropping or involuntarily throwing objects.

Clonic seizures are repetitive, rhythmic jerks that involve both sides of the body at the same time.

Tonic seizures are characterized by stiffening of the muscles.

Atonic seizures consist of a sudden and general loss of muscle tone, particularly in the arms and legs, which often results in a fall.

Seizures described herein can include epileptic seizures; acute repetitive seizures; cluster seizures; continuous seizures; unremitting seizures; prolonged seizures; recurrent seizures; status epilepticus seizures, e.g., refractory convulsive status epilepticus, non-convulsive status epilepticus seizures; refractory seizures; myoclonic seizures; tonic seizures; tonic-clonic seizures; simple partial seizures; complex partial seizures; secondarily generalized seizures; atypical absence seizures; absence seizures; atonic seizures; benign Rolandic seizures; febrile seizures; emotional seizures; focal seizures; gelastic seizures; generalized onset seizures; infantile spasms; Jacksonian seizures; massive bilateral myoclonus seizures; multifocal seizures; neonatal onset seizures; nocturnal seizures; occipital lobe seizures; post traumatic seizures; subtle seizures; Sylvan seizures; visual reflex seizures; or withdrawal seizures.

### Tremor

The solutions or admixtures described herein can be used in a method described herein, for example in the treatment of a disorder described herein such as tremor.

Tremor is an involuntary, at times rhythmic, muscle contraction and relaxation that can involve oscillations or twitching of one or more body parts (e.g., hands, arms, eyes, face, head, vocal folds, trunk, legs).

Cerebellar tremor or intention tremor is a slow, broad tremor of the extremities that occurs after a purposeful movement. Cerebellar tremor is caused by lesions in or damage to the cerebellum resulting from, e.g., tumor, stroke, disease (e.g., multiple sclerosis, an inherited degenerative disorder).

Dystonic tremor occurs in individuals affected by dystonia, a movement disorder in which sustained involuntary muscle contractions cause twisting and repetitive motions and/or painful and abnormal postures or positions. Dystonic tremor may affect any muscle in the body. Dystonic tremors occurs irregularly and often can be relieved by complete rest.

Essential tremor or benign essential tremor is the most common type of tremor. Essential tremor may be mild and nonprogressive in some, and may be slowly progressive, starting on one side of the body but affect both sides within 3 years. The hands are most often affected, but the head, voice, tongue, legs, and trunk may also be involved. Tremor frequency may decrease as the person ages, but severity may increase. Heightened emotion, stress, fever, physical exhaustion, or low blood sugar may trigger tremors and/or increase their severity.

Orthostatic tremor is characterized by fast (e.g., greater than 12 Hz) rhythmic muscle contractions that occurs in the legs and trunk immediately after standing. Cramps are felt in the thighs and legs and the patient may shake uncontrollably when asked to stand in one spot. Orthostatic tremor may occurs in patients with essential tremor.

Parkinsonian tremor is caused by damage to structures within the brain that control movement. Parkinsonian tremor is often a precursor to Parkinson's disease and is typically seen as a “pill-rolling” action of the hands that may also affect the chin, lips, legs, and trunk. Onset of parkinsonian tremor typically begins after age 60. Movement starts in one limb or on one side of the body and can progress to include the other side.

Physiological tremor can occur in normal individuals and have no clinical significance. It can be seen in all voluntary muscle groups. Physiological tremor can be caused by certain drugs, alcohol withdrawl, or medical conditions including an overactive thyroid and hypoglycemia. The tremor classically has a frequency of about 10 Hz.

Psychogenic tremor or hysterical tremor can occur at rest or during postural or kinetic movement. Patient with psychogenic tremor may have a conversion disorder or another psychiatric disease.

Rubral tremor is characterized by coarse slow tremor which can be present at rest, at posture, and with intention. The tremor is associated with conditions that affect the red nucleus in the midbrain, classical unusual strokes.

### Anesthesia/Sedation

The solutions or admixtures described herein can be used in a method described herein, for example to induce anesthesia or sedation. Anesthesia is a pharmacologically induced and reversible state of amnesia, analgesia, loss of responsiveness, loss of skeletal muscle reflexes, decreased stress response, or all of these simultaneously. These effects can be obtained from a single drug which alone provides the correct combination of effects, or occasionally with a combination of drugs (e.g., hypnotics, sedatives, paralytics, analgesics) to achieve very specific combinations of results. Anesthesia allows patients to undergo surgery and other procedures without the distress and pain they would otherwise experience.

Sedation is the reduction of irritability or agitation by administration of a pharmacological agent, generally to facilitate a medical procedure or diagnostic procedure.

Sedation and analgesia include a continuum of states of consciousness ranging from minimal sedation (anxiolysis) to general anesthesia.

Minimal sedation is also known as anxiolysis. Minimal sedation is a drug-induced state during which the patient responds normally to verbal commands Cognitive function and coordination may be impaired. Ventilatory and cardiovascular functions are typically unaffected.

Moderate sedation/analgesia (conscious sedation) is a drug-induced depression of consciousness during which the patient responds purposefully to verbal command, either alone or accompanied by light tactile stimulation. No interventions are usually necessary to maintain a patent airway. Spontaneous ventilation is typically adequate. Cardiovascular function is usually maintained.

Deep sedation/analgesia is a drug-induced depression of consciousness during which the patient cannot be easily aroused, but responds purposefully (not a reflex withdrawal from a painful stimulus) following repeated or painful stimulation. Independent ventilatory function may be impaired and the patient may require assistance to maintain a patent airway. Spontaneous ventilation may be inadequate. Cardiovascular function is usually maintained.

General anesthesia is a drug-induced loss of consciousness during which the patient is not arousable, even to painful stimuli. The ability to maintain independent ventilatory function is often impaired and assistance is often required to maintain a patent airway. Positive pressure ventilation may be required due to depressed spontaneous ventilation or drug-induced depression of neuromuscular function. Cardiovascular function may be impaired.

Sedation in the intensive care unit (ICU) allows the depression of patients' awareness of the environment and reduction of their response to external stimulation. It can play a role in the care of the critically ill patient, and encompasses a wide spectrum of symptom control that will vary between patients, and among individuals throughout the course of their illnesses. Heavy sedation in critical care has been used to facilitate endotracheal tube tolerance and ventilator synchronization, often with neuromuscular blocking agents.

In some embodiments, sedation (e.g., long-term sedation, continuous sedation) is induced and maintained in the ICU for a prolonged period of time (e.g., 1 day, 2 days, 3 days, 5 days, 1 week, 2 week, 3 weeks, 1 month, 2 months). Long-term sedation agents may have long duration of action. Sedation agents in the ICU may have short elimination half-life.

Procedural sedation and analgesia, also referred to as conscious sedation, is a technique of administering sedatives or dissociative agents with or without analgesics to induce a state that allows a subject to tolerate unpleasant procedures while maintaining cardiorespiratory function.

### Methods of Administration

The aqueous solution or admixture described herein comprising a therapeutically effective amount of a neuroactive steroid, a cyclodextrin, and a buffer may be administered parenterally (e.g., intranasally, buccally, intravenously or intramuscularly, for example, intramuscular (IM) injection or intravenously).

In one embodiment, the aqueous solution or admixture comprising a neuroactive steroid is administered in a dose equivalent to a parenteral administration of about 0.1 ng to about 100 g per kg of body weight, about 10 ng to about 50 g per kg of body weight, about 100 ng to about 1 g per kg of body weight, from about 1 μg to about 100 mg per kg of body weight, from about 10 μg to about 10 mg per kg of body weight, from about 100 μg to about 5 mg per kg of body weight, from about 250 μg to about 3 mg per kg of body weight, from about 500 μg to about 2 mg per kg of body weight, from about 1 μg to about 50 mg per kg of body weight, from about 1 μg to about 500 μg per kg of body weight; and from about 1 μg to about 50 μg per kg of body weight of the neuroactive steroid. Alternatively, the amount of aqueous solution or admixture comprising a neuroactive steroid administered to achieve a therapeutic effective dose is about 0.1 ng, 1 ng, 10 ng, 100 ng, 1 μg, 10 μg, 100 μg, 1 mg, 1.5 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 30 mg, 40 mg, 50 mg, 60 mg, 70 mg, 80 mg, 90 mg, 100 mg, 500 mg per kg of body weight or greater of the neuroactive steroid.

In one embodiment, the aqueous solution or admixture comprising a neuroactive steroid is administered as an intravenous bolus infusion in a dose equivalent to parenteral administration of about 0.1 ng to about 100 g per kg of body weight, about 10 ng to about 50 g per kg of body weight, about 100 ng to about 1 g per kg of body weight, from about 1 μg to about 100 mg per kg of body weight, from about 1 μg to about 50 mg per kg of body weight, from about 10 μg to about 5 mg per kg of body weight, from about 100 μg to about 500 μg per kg of body weight, from about 100 μg to about 400 μg per kg of body weight, from about 150 μg to about 350 μg per kg of body weight, from about 250 μg to about 300 μg per kg of body weight of the neuroactive steroid. In one embodiment, the aqueous solution or admixture comprising a neuroactive steroid is administered as an intravenous bolus infusion in a dose equivalent to parenteral administration of about 100 to about 400 μg/kg of the neuroactive steroid. In some embodiments, the aqueous solution or admixture comprising a neuroactive steroid is administered as an intravenous bolus infusion at about 150 to about 350 μg/kg of the neuroactive steroid. In some embodiments, the aqueous solution or admixture comprising a neuroactive steroid is administered as an intravenous bolus infusion at about 250 to about 300 μg/kg of the neuroactive steroid. In specific embodiments, the aqueous solution or admixture comprising a neuroactive steroid is administered as an intravenous bolus infusion in a dose equivalent to about 100 μg/kg, 125 μg/kg, 150 μg/kg, 175 μg/kg, 200 μg/kg, 225 μg/kg, 250 μg/kg, 260 μg/kg, 270 μg/kg, 280 μg/kg, 290 μg/kg, 300 μg/kg, 325 μg/kg, or 350 μg/kg of the neuroactive steroid.

In one embodiment, the aqueous solution or admixture comprising a neuroactive steroid is administered as an intravenous bolus infusion in a dose equivalent to parenteral administration of about 0.1 nmoles/L to about 100 μmoles/L per kg of body weight, about 1 nmoles/L to about 10 μmoles/L per kg of body weight, about 10 nmoles/L to about 10 μmoles/L per kg of body weight, about 100 nmoles/L to about 10 μmoles/L per kg of body weight, about 300 nmoles/L to about 5 μmoles/L per kg of body weight, about 500 nmoles/L to about 5 μmoles/L per kg of body weight, and about 750 nmoles/L to about 1 μmoles/L per kg of body weight of the neuroactive steroid. Alternatively, the amount of aqueous solution or admixture comprising a neuroactive steroid administered to achieve a therapeutic effective dose is about 0.1 ng, 1 ng, 10 ng, 100 ng, 1 μg, 10 μg, 100 μg, 1 mg, 1.5 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 40 mg, 50 mg, 60 mg, 70 mg, 80 mg, 90 mg, 100 mg, 500 mg per kg of body weight or greater of the neuroactive steroid.

In some embodiments, the aqueous solution or admixture comprising a neuroactive steroid may be administered once or several times a day. A duration of treatment may follow, for example, once per day for a period of about 1, 2, 3, 4, 5, 6, 7 days or more. In some embodiments, either a single dose in the form of an individual dosage unit or several smaller dosage units or by multiple administrations of subdivided dosages at certain intervals is administered. For instance, a dosage unit can be administered from about 0 hours to about 1 hr, about 1 hr to about 24 hr, about 1 to about 72 hours, about 1 to about 120 hours, or about 24 hours to at least about 120 hours post injury. Alternatively, the dosage unit can be administered from about 0.5, 1, 1.5, 2, 3, 4, 5, 6, 7, 8, 9, 10, 11, 12, 13, 14, 15, 16, 17, 18, 19, 20, 21, 22, 23, 24, 30, 40, 48, 72, 96, 120 hours or longer post injury. Subsequent dosage units can be administered any time following the initial administration such that a therapeutic effect is achieved. For instance, additional dosage units can be administered to protect the subject from the secondary wave of edema that may occur over the first several days post-injury.

In some embodiments, the aqueous solution or admixture comprising a neuroactive steroid administration includes a time period in which the administration is weaned off.

As used herein, “weaning” or “weaning dose” refers to an administration protocol which reduces the dose of administration to the patient and thereby produces a gradual reduction and eventual elimination of the aqueous solution or admixture comprising a neuroactive steroid, either over a fixed period of time or a time determined empirically by a physician's assessment based on regular monitoring of a therapeutic response of a subject. The period of the weaned administration can be about 12, 24, 36, 48 hours or longer. Alternatively, the period of the weaned administration can range from about 1 to 12 hours, about 12 to about 48 hours, or about 24 to about 36 hours. In some embodiments, the period of the weaned administration is about 24 hours.

The weaning employed could be a “linear” weaning. For example, a “10%” linear weaning from 500 mg would go 500, 450, 400, 350, 300, 250, 200, 150, 100, 50. Alternatively, an exponential weaning could be employed which, if the program outlined above is used as an example, the exponential weaning would be, e.g., 500, 450, 405, 365, 329, 296, 266, 239, etc. Accordingly, about a 5%, 10%, 15%, 20%, 25%, 30%, 35%, or 40% linear or exponential weaning could be employed in the methods of the invention. In addition, a linear or exponential weaning of about 1% to 5%, about 6% to 10%, about 11% to 15%, about 16% to 20%, about 21% to 25%, about 26% to 30%, about 31% to 35%, about 36% to 40% could be employed.

In other embodiments, the aqueous solution or admixture comprising a neuroactive steroid administration includes a final time period in which the administration of neuroactive steroid is tapered off.

As used herein, “tapered administration”, “tapered dose”, and “downward taper dose” refers to an administration protocol which reduces the dose of administration to the patient and thereby produces a gradual reduction and eventual elimination of aqueous solution or admixture comprising a neuroactive steroid, either over a fixed period of time or a time determined empirically by a physician's assessment based on regular monitoring of a therapeutic response of a subject. The period of the tapered administration can be about 12, 24, 36, 48 hours or longer. Alternatively, the period of the tapered administration can range from about 1 to 12 hours, about 12 to about 48 hours, or about 24 to about 36 hours. In some embodiments, the period of the tapered administration is about 24 hours.

The taper employed could be a “linear” taper. For example, a “10%” linear taper from 500 mg would go 500, 450, 400, 350, 300, 250, 200, 150, 100, 50 mg. Alternatively, an exponential taper could be employed which, if the program outlined above is used as an example, the exponential taper would be, e.g., 500, 450, 405, 365, 329, 296, 266, 239, etc. Accordingly, about a 5%, 10%, 15%, 20%, 25%, 30%, 35%, or 40% linear or exponential taper could be employed in the methods of the invention. In addition, a linear or exponential taper of about 1% to 5%, about 6% to 10%, about 11% to 15%, about 16% to 20%, about 21% to 25%, about 26% to 30%, about 31% to 35%, about 36% to 40% could be employed. In some embodiments, the drug taper is a about 25% linear taper.

In one embodiment, the aqueous solution or admixture comprising a neuroactive steroid is administered as an intravenous infusion at an amount of neuroactive steroid/unit time of about 20 to about 5000 μg/kg/hr. In some embodiments, the maintenance cycle the neuroactive steroid is administered as an intravenous infusion at an amount of neuroactive steroid/unit time of about 20 to about 2500 μg/kg/hr. In some embodiments, the maintenance cycle the neuroactive steroid is administered as an intravenous infusion at an amount of neuroactive steroid/unit time of about 20 to about 500 μg/kg/hr. In some embodiments, the neuroactive steroid is administered as an intravenous infusion at a rate of about 20 to about 250 μg/kg/hr. In some embodiments, the neuroactive steroid is administered as an intravenous infusion at an amount of neuroactive steroid/unit time of about 20 to about 200 μg/kg/hr. In some embodiments, the neuroactive steroid is administered as an intravenous infusion at an amount of neuroactive steroid/unit time of about 20 to about 150 μg/kg/hr. In some embodiments, the neuroactive steroid is administered as an intravenous infusion at an amount of neuroactive steroid/unit time of about 50 to about 100 μg/kg/hr. In some embodiments, the neuroactive steroid is administered as an intravenous infusion at an amount of neuroactive steroid/unit time of about 70 to about 100 μg/kg/hr. In specific embodiments, the neuroactive steroid is administered as an intravenous infusion at an amount of neuroactive steroid/unit time of about 25 μg/kg/hr, 50 μg/kg/hr, 75 μg/kg/hr, 80 μg/kg/hr, 85 μg/kg/hr, 86 μg/kg/hr, 87 μg/kg/hr, 88 μg/kg/hr, 89 μg/kg/hr, 90 μg/kg/hr, 100 μg/kg/hr, 125 μg/kg/hr, 150 μg/kg/hr, or 200 μg/kg/hr.

In one embodiment, the aqueous solution or admixture comprising a neuroactive steroid is administered as an intravenous infusion in a dose equivalent to parenteral administration of about 0.1 ng to about 100 g per kg of body weight, about 10 ng to about 50 g per kg of body weight, about 100 ng to about 1 g per kg of body weight, from about 1μg to about 100 mg per kg of body weight, from about 1 to about 50 mg per kg of body weight, from about 10 μg to about 5 mg per kg of body weight; and from about 100 μg to about 1000 μg per kg of body weight of the neuroactive steroid. In one embodiment, the aqueous solution or admixture comprising a neuroactive steroid is administered as an intravenous infusion in a dose equivalent to parenteral administration of about 0.1 nmoles/L to about 100 μmoles/L per kg of body weight, about 1 nmoles/L to about 10 μmoles/L per kg of body weight, about 10 nmoles/L to about 10 μmoles/L per kg of body weight, about 100 nmoles/L to about 10 μmoles/L per kg of body weight, about 300 nmoles/L to about 5 μmoles/L per kg of body weight, about 500 nmoles/L to about 5 μmoles/L per kg of body weight, and about 750 nmoles/L to about 5 μmoles/L per kg of body weight of the neuroactive steroid. Alternatively, the amount of aqueous solution or admixture comprising a neuroactive steroid administered to achieve a therapeutic effective dose is about 0.1 ng, 1 ng, 10 ng, 100 ng, 1 μg, 10 μg, 100 μg, 1 mg, 1.5 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 40 mg, 50 mg, 60 mg, 70 mg, 80 mg, 90 mg, 100 mg, 500 mg of the neuroactive steroid per kg of body weight or greater.

As used herein, “about” means approximately plus or minus ten percent.

### EXAMPLES

### Example 1. Degradation Pathway for Allopregnanolone in SBECD Formulations

FIG. 1 summarizes the two major degradation pathways found for allopregnanolone in SBECD formulations. Based on data described in FIGS. 3-5 and FIG. 8 and Tables 1-11 and Table 16, the major degradation pathway observed at a pH of 6 or less is epimerization of allopregnanolone to compound 1269. Based on data described in FIGS. 3-5 and FIG. 8 and Tables 1-11 and Table 16, the major degradation pathway observed at a pH of 6 or more is oxidation of allopregnanolone to compound 136.

Solubility of allopregnanolone was determined in sulfobutylether-β-cyclodextrin without a buffer. The graphical depiction of allopregnanolone as a function of cyclodextrin is shown in FIG. 2.

### Example 2. Allopregnanolone in Sulfobutylether-β-Cyclodextrin without a Buffer

A formulation of allopregnanolone (5 mg/mL) in 250 mg/mL sulfobutylether-β-cyclodextrin was prepared without a buffer, and packaged in a Type I glass vial.

Specifically, the formulation was manufactured by dissolving the required amount of Betadex Sulfobutyl Ether Sodium (i.e., sulfobutylether-β-cyclodextrin) in approximately 80% of the required amount of Sterile Water for Injection (SWI) in a suitable vessel with a standard impeller agitator at 35-40° C. Allopregnanolone was added to the un-buffered Betadex Sulfobutyl Ether Sodium (i.e., sulfobutylether-β-cyclodextrin) solution and mixed to dissolve with a high shear agitator. High shear mixing at 35-40° C. was continued until the solution was visibly clear, indicating that the allopregnanolone drug substance was dissolved. The bulk solution was brought to final volume with SWI and mixed. The solution was filtered through a 0.45 μm pre-filter and aseptically filtered through suitably redundant sterile 0.2 μm filters (such as a Millipore PVDF) into a previously sterilized filling vessel. The sterile solution was aseptically filled into previously sterilized vials, sealed with previously sterilized stoppers and the stoppers affixed to the vials with crimped aluminum seals. The filled vials were 100% inspected for visible particulates and container closure defects, sampled for release testing and stored at 2-8° C.

The stability results indicated a downward drift in pH and evidence of degradation (formation of compound 136 and 1269), which was faster at higher temperatures. The presence of degradation products at higher temperatures render the allopregnanolone formulation chemically unstable at these conditions. The unstable formulation limits the usable timeframe for the materials in human clinical trials and potential commercial applications.

In Table 1, formulations of allopregnanolone (5 mg/mL) in 250 mg/mL sulfobutylether-β-cyclodextrin without a buffer were monitored for 9 months at 25° C./60% RH. The pH, assay, amount of impurities and particulate matter were recorded.

### Formulation Stability

TABLE 1
Formulation of 5 mg/mL of allopregnanolone in 250 mg/mL SBECD, 20 mL vials,
unbuffered un-autoclaved and stored at 25° C./60% RH for 9 months

| Test | | Initial | 1 Month | 3 Month | 4 Month | 6 Month | 7 Month | 9 Month |
|---|---|---|---|---|---|---|---|---|
| Appearance | | Conforms | Conforms | Conforms | Conforms | Conforms | Conforms | Conforms |
| pH | | 5.4 | 5.4 | 4.8 | 4.5 | 4.3 | 4.1 | 4.1 |
| Assay | | 102.7 | 102.2 | 101.8 | 101.2 | 101.0 | 102.2 | 100.8 |
| Related Substances | 136 | ND | 0.17 | 0.44 | 0.56 | 0.74 | 0.93 | 1.26 |
| by HPLC Known | 1269 | ND | ND | ND | ND | 0.14 | 0.14 | 0.20 |
| Impurities¹ (area %) | | | | | | | | |
| Particulate Matter | Number ≥10 μm: | 35 | 11 | 48 | 22 | 52 | 49 | NT |
| | Number ≥25 μm: | 7 | 0 | 16 | 0 | 3 | 5 | |

In Table 2, formulations of allopregnanolone (5 mg/mL) in 250 mg/mL sulfobutylether-β-cyclodextrin without a buffer were monitored for 3 months at 40° C./75% RH. The pH, assay, amount of impurities and particulate matter were recorded.

TABLE 2
Formulation of 5 mg/mL of allopregnanolone in 250 mg/mL
SBECD, 20 mL vials, unbuffered un-autoclaved and stored
at 40° C./75% RH for 3 months

| Test | | Initial | 1 Month | 3 Month |
|---|---|---|---|---|
| Appearance | | Conforms | Conforms | Conforms |
| pH | | 5.4 | 5.4 | 4.7 |
| Assay | | 102.7 | 101.4 | 99.9 |
| Related | 136 | ND | 0.58 | 2.87 |
| Substances by | 1269 | ND | 0.10 | 0.42 |
| HPLC | | | | |
| Known | | | | |
| Impurities | | | | |
| (area %) | | | | |
| Particulate | Number ≥ 10 μm | 35 | 23 | 51 |
| Matter | Number ≥ 25 μm | 7 | 1 | 3 |

In Table 3, formulations of allopregnanolone (5 mg/mL) in 250 mg/mL sulfobutylether-β-cyclodextrin without a buffer were monitored for 6 months at 40° C./75% RH. The pH, assay, amount of impurities and particulate matter were recorded.

TABLE 3
Formulation of 5 mg/mL of allopregnanolone in 250 mg/mL
SBCED, 20 mL vials, unbuffered un-autoclaved and stored
at 40° C./75% RH for 6 months

| Test | | Initial | 1 Month | 3 Month | 6 Month |
|---|---|---|---|---|---|
| Appearance | | Conforms | Conforms | Conforms | Conforms |
| pH | | 5.6 | 5.2 | 4.9 | 4.3 |
| Assay | | 98.2 | 98.0 | 98.3 | 96.9 |
| Related | 136 | ND | 0.15 | 0.31 | 0.57 |
| Substances | 1269 | ND | ND | 0.14 | 0.48 |
| by HPLC | | | | | |
| Known | | | | | |
| Impurities | | | | | |
| (area %) | | | | | |
| Particulate | Number ≥ | 115 | 80 | 80 | 78 |
| Matter | 10 μm | | | | |
| | Number ≥ | 3 | 7 | 11 | 4 |
| | 25 μm | | | | |

### Example 3. Allopregnanolone in Sulfobutylether-β-Cyclodextrin with a Buffer

A formulation of allopregnanolone (5 mg/mL) in 250 mg/mL sulfobutylether-β-cyclodextrin was prepared with a citrate buffer, and packaged in a Type I glass vial.

Seven allopregnanolone solutions were prepared as described in Table 4. Batches of each of the seven solutions were autoclaved at 121° C. for 30, 60 and 90 minutes. The solutions were stored at room temperature prior to testing. Table 5 summarizes the initial pH values for the solutions.

TABLE 4
Compositions of Allopregnanolone Formulation Prepared for Testing

| | | 5 mM | 5 mM | 5 mM | 10 mM | 10 mM | 10 mM |
|---|---|---|---|---|---|---|---|
| Component | Control | pH 5.5 | pH 6.0 | pH 6.5 | pH 5.5 | pH 6.0 | pH 6.5 |
| Allo (g/L) | 5 | | | | | | |
| Citric Acid | NA | 0.25 | 0.13 | 0.05 | 0.51 | 0.27 | 0.11 |
| Monohydrate (g/L) | | | | | | | |
| Sodium Citrate | NA | 1.12 | 1.28 | 1.40 | 2.23 | 2.57 | 2.79 |
| dihydrate (g/L) | | | | | | | |

| | |
|---|---|
| Sodium | To adjust pH |
| Hydroxide or | |
| Citric Acid QS | |
| Captisol ® (g/L) | 250 |
| WFI | QS to 1 L |

TABLE 5
pH Summary of Initial Buffer Formulations
Buffer Preparation

| | | pH after | |
|---|---|---|---|
| | | addition of | |
| Buffer | | citric | |
| Concentration | | acid/sodium | Final Adjusted |
| (mM) | Target pH | citrate | pH |
| 5 | 5.5 | 5.73 | 5.50 |
| 5 | 6.0 | 6.24 | 5.96 |
| 5 | 6.5 | 6.40 | 6.50 |
| 10 | 5.5 | 5.62 | 5.51 |
| 10 | 6.0 | 6.12 | 6.02 |
| 10 | 6.5 | 6.60 | 6.60 |

A comparison of the assay values for the un-autoclaved and autoclaved samples are shown in Table 6. The data indicates that the assay value (%) held steady for all autoclave times studied.

TABLE 6
Effect of Autoclaving on Product Assay

| | Target | Assay Following Autoclaving (121° C.) | | | |
|---|---|---|---|---|---|
| Prototype | pH | Initial Assay* | 30 min | 60 min | 90 min |
| Control | N/A | 101.0 | 99.9 | 100.4 | 99.5 |
| Buffered 5 mM | 5.5 | 101.9 | 101.4 | 101.2 | 100.6 |
| | 6.5 | 101.3 | 101.6 | 102.6 | 101.4 |
| Buffered 10 mM | 5.5 | 101.8 | 100.0 | 99.2 | 99.7 |
| | 6.5 | 100.6 | 99.9 | 100.3 | 99.5 |

*Assay of Time Zero, non-autoclaved sample.

Table 7 summarizes the impurity profile, specifically the amounts of compounds 136 and 1269 formed during the brief exposure to high temperatures.

An oxidative degradant (Compound 136) was observed at a level of 0.13% after autoclaving in the control sample for 90 minutes. Similar levels were found in the 90 minute pH 5.5 buffered samples (0.15% for the 5 mM sample and 0.11% for the 10 mM sample). The 90 minute pH 6.5 buffered samples contained a slightly lower level of the oxidative degradant (0.05% for the 5 mM sample and 0.03% for the 10 mM sample).

Under the conditions of this study, there is a noticeable and slight improvement in the lack of formation of the compound 136 with the 10 mM buffer compared to the 5 mM buffer.

Importantly, no epimerization (formation of compound 1269) is observed during the heat stress study, compare (Not Detected (ND) for the buffered formulations versus 0.15% at the 90 minute timepoint for the un-buffered control.

TABLE 7
Impurity Profile after Autoclaving

Impurity Profile After Autoclaving
for 30, 60 or 90 minutes

| | | | | |
|---|---|---|---|---|
| | Target | | | |
| Prototype | pH | 30 min | 60 min | 90 min |

Control

| | | | | |
|---|---|---|---|---|
| 136 | N/A | 0.10 | 0.10 | 0.13 |
| 1269 | | ND | 0.11 | 0.15 |

Buffered 5 mM

| | | | | |
|---|---|---|---|---|
| 136 | 5.5 | 0.06 | 0.11 | 0.15 |
| 1269 | | ND | ND | ND |
| 136 | 6.5 | 0.02 | 0.03 | 0.05 |
| 1269 | | ND | ND | ND |

Buffered 10 mM

| | | | | |
|---|---|---|---|---|
| 136 | 5.5 | 0.04 | 0.08 | 0.11 |
| 1269 | | ND | ND | ND |
| 136 | 6.5 | 0.01 | 0.02 | 0.03 |
| 1269 | | ND | ND | ND |

Table 8 summarizes the initial pH, initial assay and impurity data for each batch. The table includes un-autoclaved control samples along with the initial T=0 autoclaved samples. Samples were analyzed for pH at approximately 3 months storage at room temperature conditions, and for assay and impurities after approximately 4 months at room temperature.

TABLE 8
Summary of Initial Assay and Impurities
Data - Autoclaved versus Un-autoclaved

| | | Initial | Total | | | Total |
|---|---|---|---|---|---|---|
| | Initial | Assay | Impurities | | Assay | Impurities |
| Description | pH | % LC | % Area | pH | % LC | % Area |
| Control | 5.7 | 101.1 | 0.79 | 5.1 | 101.2 | 0.93 |
| autoclaved | | | | | | |
| Control | 6.3 | 101.0 | 0.83 | 5.2 | 101.4 | 1.10 |
| not autoclaved | | | | | | |
| 5 mM, pH 5.5 | 5.1 | 101.6 | 0.84 | 5.0 | 101.5 | 0.94 |
| autoclaved | | | | | | |
| 5 mM, pH 6.0 | 5.9 | ** | ** | 5.4 | 102.0 | 1.25 |
| autoclaved | | | | | | |
| 5 mM, pH 6.5 | 5.5 | 102.9 | 0.85 | 5.9 | 102.3 | 1.02 |
| autoclaved | | | | | | |
| 10 mM, pH 5.5 | 5.1 | 101.5 | 0.83 | 5.1 | 100.6 | 0.84 |
| autoclaved | | | | | | |
| 10 mM, pH 6.0 | 5.5 | ** | ** | 5.6 | 99.6 | 0.84 |
| autoclaved | | | | | | |
| 10 mM, pH 6.5 | 6.1 | 100.7 | 0.85 | 6.1 | 100.2 | 0.84 |
| autoclaved | | | | | | |
| 5 mM, pH 5.5 | 5.1 | 101.9 | 0.84 | 5.1 | 101.1 | 0.84 |
| Not autoclaved | | | | | | |
| 5 mM, pH 6.0 | 5.4 | ** | ** | 5.4 | 102.1 | 1.04 |
| Not autoclaved | | | | | | |
| 5 mM, pH 6.5 | 5.9 | 101.3 | 0.85 | 5.9 | 102.4 | 0.96 |
| Not autoclaved | | | | | | |
| 10 mM, pH 5.5 | 5.1 | 101.8 | 0.88 | 5.1 | 100.4 | 0.84 |
| Not autoclaved | | | | | | |
| 10 mM, pH 6.0 | 5.5 | ** | ** | 5.5 | 99.8 | 0.84 |
| Not autoclaved | | | | | | |
| 10 mM, pH 6.5 | 6.1 | 100.6 | 0.84 | 6.1 | 100.9 | 0.97 |
| Not autoclaved | | | | | | |

** Not tested at initial time point

The pH of the un-buffered, non-autoclaved control sample dropped 1.1 pH units after 3 months of storage at room temperature and the pH of the un-buffered, autoclaved control sample dropped 0.6 pH units after 3 months of storage at room temperature

The pH of the buffered solutions did not change significantly (the largest pH change reported was 0.1 pH units).

Both the 5 and 10 mM buffer concentrations provided good pH control after autoclaving and storage.

The initial data (T=0) for assay (%) and total impurities across the prototypes indicated a consistent range from 100.6-102.9% and 0.79-0.85%, respectively. The assay values in the T=4 months samples were consistent with the T=0 samples and showed no indication of any degradation. This was also the case for the total impurities.

Larger lots of a formulation of allopregnanolone (5 mg/mL) in 250 mg/mL sulfobutylether-β-cyclodextrin were prepared with a citrate buffer, and packaged in a Type I glass vial.

Specifically, the formulation was manufactured by dissolving the required amount of citric acid monohydrate (USP) and sodium citrate dihydrate (USP) in approximately 80% of the required amount of Sterile Water for Injection (SWI) in a suitable vessel with a standard impeller agitator at 35-40° C. The required amount of Betadex Sulfobutyl Ether Sodium (i.e., sulfobutylether-β-cyclodextrin) was added to the buffer solution and mixed to dissolve. The product pH was checked and adjusted, if required, with hydrochloric acid or sodium hydroxide to a pH of 6.0+/−0.2. Allopregnanolone was added to the buffered Betadex Sulfobutyl Ether Sodium (i.e., sulfobutylether-β-cyclodextrin) solution and mixed to dissolve with a high shear agitator. High shear mixing at 35-40° C. was continued until the solution was visibly clear, indicating that the allopregnanolone drug substance was dissolved. The product pH was checked and adjusted, if required, with hydrochloric acid or sodium hydroxide to ensure that the product had a pH of 6.0+/−0.1. The bulk solution was brought to final volume with SWFI and mixed. The solution was filtered through a 0.45 inn pre-filter and aseptically filtered through suitably redundant sterile 0.2 μm filters (such as a Millipore PVDF) into a previously sterilized filling vessel. The sterile solution was aseptically filled into previously sterilized vials, sealed with previously sterilized stoppers and the stoppers affixed to the vials with crimped aluminum seals (component described in Table 9). The filled vials were 100% inspected for visible particulates and container closure defects, sampled for release testing and stored at 2-8° C.

TABLE 9
Packaging Configuration for Formulations

| | | |
|---|---|---|
| Vial | Vial specification number | PC 3196 |
| | Vial Description | USP Type I Borosilicate glass |
| | | 20 mL vial with 20 mm opening |
| | Manufacturer | Schott |
| Stopper | Stopper Specification | PC4078 |
| | Number | |
| | Stopper Description | S10-F451 Chlorobutyl B2-40 |
| | | Coating, FluroTec |
| | | Item 19700021 or 19700022 |
| | Manufacturer | West |
| Overseal | Seal Description | Aluminum Seal, 20 mm |
| | Manufacturer | West Pharmaceutical Services |
| | Seal Color | N/A* |

*Non-product contact. Different seal colors were used to distinguish the formulation differences.

In Table 10, formulations of allopregnanolone (5 mg/mL) in 250 mg/mL sulfobutylether-β-cyclodextrin in 10 mM citrate buffer pH 6 were monitored for 6 months at 40° C./75% RH. The pH, assay (e.g., percent label claim), amount of impurities and particulate matter were recorded.

TABLE 10
Injection 5 mg/mL of allopregnanolone in 250 mg/mL
SBECD, 20 mL vials, 10 mM citrate buffer pH = 6,
stored at 40° C./75% RH for 6 months

| Test | | Initial | 1-Month | 3-Month | 6-Month |
|---|---|---|---|---|---|
| Appearance | | Conforms | Conforms | Conforms | Conforms |
| pH | | 5.8 | 5.7 | 5.8 | 5.9 |
| Assay (%) | | 99.5 | 98.8 | 99.0 | 98.4 |
| Related | 136 | ND | ND | ND | <0.10 |
| Substances | | | | | |
| by HPLC | | | | | |
| (wt %) | | | | | |
| | 1269 | <0.10 | <0.10 | <0.10 | 0.12 |
| Particulate | ≥10 μm | 76 | 163 | 319 | 38 |
| Matter | ≥25 μm | 7 | 0 | 12 | 1 |

In Table 11, formulations of allopregnanolone (5 mg/mL) in 250 mg/mL sulfobutylether-β-cyclodextrin in 10 mM citrate buffer pH 6 were monitored for 12 months at 25° C./60% RH. The pH, assay (percent label claim), amount of impurities and particulate matter were recorded.

TABLE 11
Injection 5 mg/mL of allopregnanolone in 250 mg/mL SBECD, 20 mL vials, 10 mM
citrate buffer pH = 6, stored at 25° C./60% RH for 12 months

| Test | | Initial | 1-Month | 3-Month | 6-Month | 9-Month | 12-Month |
|---|---|---|---|---|---|---|---|
| Appearance | | Conforms | Conforms | Conforms | Conforms | Conforms | Conforms |
| pH | | 5.8 | 5.7 | 5.8 | 5.8 | 5.8 | 5.8 |
| Assay (%) | | 99.5 | 99.5 | 99.6 | 97.6 | 98.6 | 99.3 |
| Related Substances | 136 | ND | ND | ND | ND | ND | ND |
| by HPLC (wt %) | 1269 | <0.10 | <0.10 | <0.10 | 0.10 | <0.10 | 0.10 |
| Particulate Matter | ≥10 μm | 76 | 89 | 69 | 66 | 37 | 40 |
| | ≥25 μm | 7 | 0 | 1 | 18 | 1 | 4 |

### Example 4. Terminal Sterilization of Allopregnanolone for Injection, 5 mg/mL in 250 mg/mL Cyclodextrin (10 mM Citrate Buffer, pH 6.0) in 20 mL Vials

Experiments were performed to demonstrate that the sterilization process for Allopregnanolone Injection, 5 mg/mL in 250 mg/mL Captisol® (10 mM citrate buffer, pH=6.0, 20 mL/vial) provides temperature uniformity and biological kill throughout the load using the Finn-Aqua steam sterilizer, including demonstration that no growth of a known microbial load of Geobacillus stearothermophilus.

The protocol defined and validated the sterilization process and determined where the sterilizer load probes would be placed during routine operation of the product. There were three (3) maximum load sterilizer runs and three (3) minimum load sterilizer runs for each vial size using the Finn-Aqua steam sterilizer, Model 91515-DP-RP-GMP-S7, Serial No. C0A41043. The Finn-Aqua steam sterilizer was a double door unit controlled by a Siemens Simatic S7-300 Programmable Logic Controller (PLC). The sterilizer was operated from the user interface, Operator Panel OP27. The internal chamber dimensions were (w×h×d) 37 in ×61 in ×61 in, for a total internal volume of 75 cu. ft. There was a single cart, which could be outfitted with up to 15 shelves. Each shelf accommodated 8 trays of vials (each tray accommodated 162-20 mL vials). “D-value” refers to the time required at temperature (T) reduce a specific microbial population by 90%, or, as the time required for the number of survivors to be reduced by a factor of 10 (1 log).

The maximum autoclave batch size of 259 L accommodated approximately 12,690 vials. The minimum validation load was 3 L, based on minimum autoclave batch size of a single tray.

The product was aseptically filled within the sterile core of a manufacturing facility, which was supported by aseptic process simulation (media fills). These evaluations of the aseptic process validated that the product had a sterility assurance level (SAL) of 10³. Bioburden was measured in samples taken post filling and prior to terminal sterilization. It was anticipated to measure zero (0) CFU/10 mL with and alert level of >1 CFU/10 mL.

The validation was run using a dwell time equal to the proposed standard dwell time to demonstrate the process' ability to perform an 8 log reduction of the spore challenge (6 logs+a 2-log safety factor). The product D-value had been determined to be 3.5 minutes for the 20 mL vial and 4.5 for the 50 mL vial. In order to align both vial sizes with one cycle, the highest D-value was chosen. Assuming that complete kill of the BI requires 6 logs of reduction, the resulting proposed exposure (kill) time for the validation cycle would be:

tₖᵢₗₗ=D*[(log N₀)+2]=4.5*[log(5×10⁶)+2]=39.15 min

As such, the validation cycle was determined to be:
Proposed exposure: Exp Time (min): 40 min Temp: 122.2° C. ±1.0° C.

Calculated time that results in a decimal was rounded to the next minute. Additionally, in order to maintain product temperature above 121.1° C. for sterilization, the sterilizer set point during exposure was 122.2° C.

Efficacy of the terminal sterilization process was determined by temperature uniformity and demonstration of at least a 6-log reduction of the viable spore count of G. stearothermophilus, spiked at 1×10⁶ to 5×10⁶ spores per vial. Based upon successful demonstration of biological kill during the validation cycle, the production cycle exposure time would have an exposure time of 40 minutes (at the validated exposure temperature of 122.2° C.±1.0° C.), to correspond with the calculated required exposure time of the inoculated product determined during the D-value. For the 20 mL vial size, three (3) experimental full load sterilizer runs were executed that consisted of a 10 minute, 15 minute, and 20 minute exposure time. Once these three (3) experimental runs had been completed, the optimum run was chosen and verified by executing an additional two (2) sterilizer runs.

The validation consisted of two parts. Three (3) maximum load sterilizer runs were conducted with temperature-measuring devices and biological indicators distributed throughout the chamber with an emphasis on locations determined from empty chamber cycles performed during the annual autoclave re-qualification (biological indicator locations will be placed in the same location for each cycle). Three (3) minimum load sterilizer cycles were run using one (1) tray located on the top shelf of the chamber (the sterilizer was consistently loaded from the top shelf of the chamber; therefore, any sterilizer loads with less than the maximum number of trays would always have trays on the sterilizer's top shelf).

A Biological Indicator (BI) was placed next to each load probe check/penetration Probe (LPC/PP). The term probe as used in this section refers to the temperature-measuring device. All the penetration probes, sterilizer load probes, load probe check probes and Biological Indicators were placed in vials containing the product formulation; the rest of the load was composed of vials containing an equivalent amount of water. The use of the water vials was acceptable because the product formulation was an aqueous solution and its thermal properties were essentially identical to pure water.

### Challenge Test Minimum and Maximum Chamber Load

Objective: To demonstrate temperature uniformity and biological kill throughout the vial load.

Acceptance Criteria:

1) All exposed Biological Indicators (BIs) must not show growth.

2) All positive controls must show growth at the end of incubation.

3) All negative controls must test negative for growth at the end of incubation.

4) All Penetration Probes and Load-probe-check Probes should maintain a temperature range of 122.2° C.±1.0° C. during exposure.

### Example 5. Characterization Data for Compound 1269

¹H and ¹³C NMR Assignments for 1269 are provided in Table 12.

TABLE 12
¹H and ¹³C NMR Assignments for 1269 (CDCl₃)

| Structure | 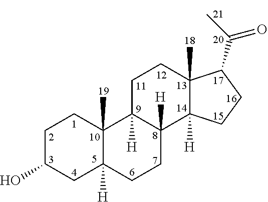 | | | | | |
|---|---|---|---|---|---|---|
| | δ_{H} | Multiplicity¹ | Proton | δ_{C} | gHSQC | gHMBC |
| Position | (ppm) | J_{H} (Hz) | Count | (ppm) | ¹J Correlation | ^{2,3}J Correlation |
| 1 | 1.43 | o m | 1 H | 32.36 | 32.36-1.43 | 32.36-0.76: ³J C₁-H₁₉ |
| | 1.27 | o m | 1 H | | 32.36-1.27 | |
| 2 | 1.64 | o m | 2 H | 29.17 | 29.17-1.64 | — |
| 3 | 4.02 | br quintet, J = 2.4 Hz | 1 H | 66.64 | 66.64-4.02 | — |
| 3a | 1.30 | br s | 1 H | — | — | — |
| 4 | 1.50 | o m | 1 H | 36.08 | 36.08-1.50 | — |
| | 1.38 | o m | 1 H | | 36.08-1.38 | |
| 5 | 1.50 | o m | 1 H | 39.16 | 39.16-1.50 | 39.16-0.76: ³J C₅-H₁₉ |
| | | | | | | 39.16-1.43: ³J C₅-H₁ |
| 6 | 1.16 | o m | 2 H | 28.72 | 28.72-1.16 | — |
| | | | | | | — |
| 7 | 1.68 | o m | 1 H | 32.41 | 32.41-1.68 | 1.01-50.55: ³J H₇-C₁₄ |
| | 1.01 | m | 1 H | | 32.41-1.01 | |
| 8 | 1.30 | o m | 1 H | 35.91 | 35.91-1.30 | — |
| 9 | 0.72 | ddd, J = 12.5, 10.2, 4.1 Hz | 1 H | 53.77 | 53.77-0.72 | 53.77-0.76: ³J C₉-H₁₉ |
| | | | | | | 53.77-1.13: ³J C₉-H₁₂ |
| 10 | — | — | — | 36.28 | — | 36.28-0.76: ²J C₁₀-H₁₉ |
| 11 | 1.58 | o m | 1 H | 20.91 | 20.91-1.58 | 20.91-1.13: ²J C₁₁-H₁₂ |
| | 1.28 | o m | 1 H | | 20.91-1.28 | 20.91-0.72: ²J C₁₁-H₉ |
| 12 | 1.73 | o m | 1 H | 35.57 | 35.57-1.73 | 35.57-2.78: ³J C₁₂-H₁₇ |
| | 1.13 | o m | 1 H | | 35.57-1.13 | 35.57-0.90: ³J C₁₂-H₁₈ |
| 13 | — | — | — | 46.01 | — | 46.01-2.78: ²J C₁₃-H₁₇ |
| | | | | | | 46.01-0.90: ²J C₁₃-H₁₈ |
| 14 | 1.25 | o m | 1 H | 50.55 | 50.55-1.25 | 50.55-0.90: ³J C₁₄-H₁₈ |
| | | | | | | 50.55-2.78: ³J C₁₄-H₁₇ |
| 15 | 1.78 | o m | 1 H | 26.06 | 26.06-1.78 | 26.06-2.78: ³J C₁₅-H₁₇ |
| | 1.20 | o m | 1 H | | 26.06-1.20 | |
| 16 | 1.90 | o m | 1 H | 24.47 | 24.47-1.90 | 24.47-2.78: ²J C₁₆-H₁₇ |
| | 1.70 | o m | 1 H | | 24.47-1.70 | |
| 17 | 2.78 | dd, J = 8.4, 2.5 Hz | 1 H | 61.59 | 61.59-2.78 | 61.59-0.90: ³J C₁₇-H₁₈ |
| | | | | | | 61.59-2.12: ³J C₁₇-H₂₁ |
| 18 | 0.90 | s | 3 H | 21.15 | 21.15-0.90 | 21.15-2.78: ³J C₁₈-H₁₇ |
| 19 | 0.76 | s | 3 H | 11.33 | 11.33-0.76 | 11.33-0.72: ³J C₁₉-H₉ |
| 20 | — | — | — | 212.96 | — | 212.96-2.12: ²J C₂₀-H₂₁ |
| | | | | | | 212.96-2.78: ²J C₂₀-H₁₇ |
| 21 | 2.12 | s | 3 H | 32.99 | 32.99-2.12 | — |

¹H chemical shifts for overlapped resonances (overlapping multiplet) are from HSQC data

LC-MS analysis of 1269 is represented in FIG. 6 and Table 13.

TABLE 13
Compound 1269 Mass Spectroscopy Assignments

| Identity | Mass |
|---|---|
| [M + H]⁺ | 303.36 |
| [M + H − H₂O]⁺ | 285.36 |
| [M + H + MeOH]⁺ | 335.41 |
| [2M + H]⁺ | 605.70 |

### Example 6. Characterization Data for Compound 136

TABLE 14
Proton and Carbon NMR Assignments for Compound 136 (CDCl₃)

| Structure | 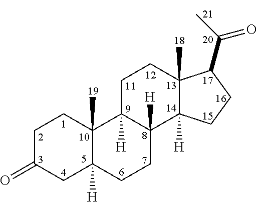 | | | | | |
|---|---|---|---|---|---|---|
| | | | | | gHSQC | gHMBC |
| | δ_{H} | Multiplicity | Proton | δ_{C} | ¹J | ^{2,3}J |
| Position | (ppm) | Jₕ (Hz) | Count | (ppm) | Correlation | Correlation |
| 1 | 2.03 | o m | 1 H | 38.73 | 38.73-2.03 | 38.73-1.02: ³J C₁-H₁₉ |
| | 1.36 | o m | 1 H | | 38.73-1.36 | |
| 2 | 2.39 | m | 1 H | 38.32 | 38.32-2.39 | — |
| | 2.30 | o m | 1 H | | 38.32-2.30 | |
| 3 | — | — | — | 212.01 | — | 212.-01-2.39: ²J C₃-H₂ |
| | | | | | | 212.-01-2.27: ²J C₃-H₄ |
| 4 | 2.27 | t, J = 14.2 Hz | 1 H | 44.83 | 44.83-2.27 | — |
| | 2.09 | o m | 1 H | | 44.83-2.09 | |
| 5 | 1.54 | m | 1 H | 46.85 | 46.85-1.55 | 46.85-1.02: ³J C₅-H₁₉ |
| | | | | | | 46.85-2.27: ³J C₅-H₄ |
| 6 | 1.32 | o m | 2 H | 29.01 | 29.01-1.32 | 29.01-1.72: ²J C₆-H₇ |
| | | | | | | 29.01-0.94: ²J C₆-H₇ |
| | | | | | | 29.01-2.27: ²J C₆-H₄ |
| 7 | 1.72 | m | 1 H | 31.83 | 31.83-1.72 | 31.83-1.17: ²J C₇-H₁₄ |
| | 0.94 | m | 1 H | | 31.83-0.94 | |
| 8 | 1.43 | o m | 1 H | 35.55 | 35.55-1.43 | — |
| 9 | 0.79 | m | 1 H | 53.85 | 53.85-0.79 | 53.85-1.02: ³J C₉-H₁₉ |
| 10 | — | — | — | 35.87 | — | 35.87-2.27: ³J C₁₀-H₄ |
| | | | | | | 35.87-1.02: ²J C₁₀-H₁₉ |
| 11 | 1.65 | o m | 1 H | 21.62 | 21.62-1.65 | 21.62-0.79: ²J C₁₁-H₉ |
| | 1.39 | o m | 1 H | | 21.62-1.39 | 21.62-2.04: ²J C₁₁-H₁₂ |
| 12 | 2.04 | o m | 1 H | 39.12 | 39.12-2.04 | 39.12-2.53: ³J C₁₂-H₁₇ |
| | 1.44 | o m | 1 H | | 39.12-1.44 | 39.12-0.64: ³J C₁₂-H₁₈ |
| 13 | — | — | — | 44.36 | — | 44.36-2.53: ²J C₁₃-H₁₇ |
| | | | | | | 44.36-0.64: ²J C₁₃-H₁₈ |
| 14 | 1.17 | o m | 1 H | 56.64 | 56.64-1.17 | 56.64-2.53: ³J C₁₄-H₁₇ |
| | | | | | | 56.64-0.64: ³J C₁₄-H₁₈ |
| 15 | 1.69 | o m | 1 H | 24.60 | 24.60-1.69 | 24.60-1.17: ²J C₁₅-H₁₄ |
| | 1.23 | o m | 1 H | | 24.06-1.39 | 24.60-1.65: ²J C₁₅-H₁₆ |
| 16 | 2.17 | m | 1 H | 23.02 | 23.02-2.17 | 23.02-2.53: ²J C₁₆-H₁₇ |
| | 1.65 | o m | 1 H | | 23.02-1.65 | 209.66-2.53: ³J H₁₆-C₂₀ |
| 17 | 2.53 | t, J = 9.0 Hz | 1 H | 63.92 | 63.92-2.53 | 63.92-0.64: ³J C₁₇-H₁₈ |
| 18 | 0.64 | s | 3 H | 13.63 | 13.63-0.64 | 13.63-2.53: ³J C₁₈-C₁₇ |
| 19 | 1.02 | s | 3 H | 11.65 | 11.65-1.02 | 11.65-0.79: ³J C₁₉-H₉ |
| 20 | — | — | — | 209.66 | — | 209.66-2.12: ²J C₂₀-H₂₁ |
| | | | | | | 209.66-2.53: ²J C₂₀-H₁₇ |
| 21 | 2.12 | s | 3 H | 31.70 | 31.70-2.12 | — |

¹H chemical shifts for overlapped resonances (o m) were taken from HSQC data.

LC-MS analysis of 136 is represented in FIG. 7 and Table 15.

TABLE 15
136 Mass Spectroscopy Assignments

| Identity | Mass |
|---|---|
| [M + H]⁺ | 317.33 |
| [M + H + CH₃CN]⁺ | 358.49 |
| [2M + H]⁺ | 633.71 |

### Example 6. pH Stability of the Allopregnanolone Formulations in SBECD

A formulation of allopregnanolone (5 mg/mL) in 250 mg/mL sulfobutylether-β-cyclodextrin was prepared at different pH values, and packaged in a Type I glass vial. The assay of the formulation was measured after 12 weeks at 40° C. (FIG. 8A). The assay of the formulation was measured after 12 weeks at 60° C. (FIG. 8B).

### Example 7. Comparison of the Effects of Different Buffers

FIGS. 3A-B depict the purity of the formulation measured after 12 weeks at 40° C. in Phosphate Buffer.
FIGS. 4A-B depict the purity of the formulation measured after 12 weeks at 40° C. in Citrate Buffer.
FIG. 5 depicts the formation of 136 over time at 40° C. and 60° C. in various buffers.

### Example 8. Stability of Allopregnanolone Formulation in Cold Temperatures

The stability of a formulation of 5 mg/mL of allopregnanolone in 250 mg/mL SBECD in 10 mM citrate buffer pH=6, was stored at 2-8° C. for 12 months. Data from the stability study is shown on Table 16.

TABLE 16
Formulation Stability for Allopregnanolone Formulation Stored at 2-8° C. for 12 months

| Test | | Initial | 1-Month | 3-Month | 6-Month | 9-Month | 12-Month |
|---|---|---|---|---|---|---|---|
| Appearance | | Conforms | Conforms | Conforms | Conforms | Conforms | Conforms |
| pH | | 5.8 | 5.7 | 5.8 | 5.8 | 5.8 | 5.8 |
| Assay (%) | | 99.5 | 99.2 | 99.1 | 98.2 | 98.8 | 97.5 |
| Related Substances | 136 | ND | ND | ND | ND | ND | ND |
| by HPLC (wt %) | 1269 | <0.10 | <0.10 | <0.10 | 0.10 | <0.10 | <0.10 |
| Particulate Matter | ≥10 μm | 76 | 69 | 38 | 214 | 25 | 163 |
| | ≥25 μm | 7 | 0 | 3 | 16 | 1 | 34 |

1. A pharmaceutically acceptable aqueous solution comprising (e.g., consisting essentially of, consisting of) a neuroactive steroid, a sulfobutyl ether beta cyclodextrin and a buffer; wherein:
the solution is a stable solution between a pH of about 3 and about 9, for at least 2 weeks; or
the buffer is present at a concentration of at least 0.1 mM; or
the solution remains substantially free of impurities for at least 2 weeks.

2. The solution of claim 1, wherein the solution is a stable solution between a pH of about 3 and about 9, for at least 2 weeks at a temperature from about 2° C. to about 8° C.

3. The solution of claim 1, wherein the solution is a stable solution between a pH of about 3 and about 9, for at least 2 weeks at a temperature from about 0° C. to about 45° C.

4. The solution of claim 1, wherein the solution remains substantially free of impurities for at least 2 weeks at a temperature from about 2° C. to about 8° C.

5. The solution of claim 1, wherein the solution remains substantially free of impurities for at least 2 weeks at a temperature from about 15° C. to about 25° C.

6. The aqueous solution of claim 1, wherein the buffer in the solution is present at a concentration of from about 5 to 100 mM.

7. The aqueous solution of claim 1, wherein the buffer in the solution is present at a concentration of from about 0.1 to about 20 mM.

8. The aqueous solution of claim 1, wherein the buffer in the solution is present at a concentration of about 0.1, about 0.5, about 1.67, or about 3.3 mM.

9. The aqueous solution of claim 1, wherein the solution is suitable for parenteral use.

10. (canceled)

11. (canceled)

12. The solution of claim 1, wherein the neuroactive steroid is allopregnanolone.

13-31. (canceled)

32. The solution of claim 1, wherein the buffer has a pKa of about 2 to about 9.

33. The solution of claim 1, wherein the buffer comprises a monoprotic acid.

34. The solution of claim 1, wherein the buffer comprises a polyprotic acid (e.g., citrate).

35. The solution of claim 1, wherein the buffer is selected from the group consisting of citrate, phosphate, acetate, lactate, gluconate, malate, succinate, tris, histidine and tartrate and mixtures thereof.

36. (canceled)

37. The solution of claim 1, wherein the buffer is selected from 4-2-hydroxyethyl-1-piperazineethanesulfonic acid (HEPES), 2-{[tris(hydroxymethyl)methyl]amino}ethanesulfonic acid (TES), 3-(N-morpholino)propanesulfonic acid (MOPS), piperazine-N,N′-bis(2-ethanesulfonic acid) (PIPES), dimethylarsinic acid (cacodylate), Citrate (e.g., saline sodium citrate), 2-(N-morpholino)ethanesulfonic acid (MES), phosphate (e.g., PBS, D-PBS), succinate (i.e., 2(R)-2-(methylamino)succinic acid), acetate, dimethylglutarate, maleate, imidazole, N-(2-Acetamido)-2-aminoethanesulfonic acid (ACES), N,N-bis(2-hydroxyethyl)-2-aminoethanesulfonic acid (BES), Bicine, Bis-Tris, Borate, N-cyclohexyl-3-aminopropanesulfonic acid (CAPS), Glycine, 3-[4-(2-Hydroxyethyl)-1-piperazinyl]propanesulfonic acid (HEPPS or EPPS), N-[Tris(hydroxymethyl)methyl]-3-aminopropanesulfonic acid, [(2-Hydroxy-1,1-bis(hydroxymethyl)ethyl)amino]-1-propanesulfonic acid (TAPS), Tricine, Tris, Tris Base, Tris Buffer, Tris-Glycine, Tris-HCl, collidine, veronal acetate, N-(2-Acetamido)iminodiacetic acid; N-(Carbamoylmethyl)iminodiacetic acid (ADA), β-Hydroxy-4-morpholinepropanesulfonic acid, 3-Morpholino-2-hydroxypropanesulfonic acid (MOPSO), cholamine chloride, 3-(N,N-Bis[2-hydroxyethyl]amino)-2-hydroxypropanesulfonic acid (DIPSO), acetamidoglycine, 3-{[1,3-Dihydroxy-2-(hydroxymethyl)-2-propanyl]amino}-2-hydroxy-1-propanesulfonic acid (TAPSO), Piperazine-N,N′-bis(2-hydroxypropanesulfonic acid) (POPSO), N-(2-Hydroxyethyl)piperazine-N′-(2-hydroxypropanesulfonic acid) (HEPPSO), N-cycloxhexyl-2-aminoethanesulfonic acid (CHES), 2-amino-methyl-1,3-proponediol (AMPd), and glycinamide.

38. (canceled)

39. (canceled)

40. The solution of claim 1, wherein the buffer is citrate.

41. The solution of claim 40, wherein the citrate buffer is present at a concentration of about 1 to about 100 mM or more.

42. The solution of claim 40, wherein the citrate buffer is present at a concentration of 5, 10, 20, 50, 100 mM or more.

43-67. (canceled)

68. A method of preparing an aqueous solution comprising a neuroactive steroid, a sulfoalkyl ether beta cyclodextrin, and a buffer, wherein the solution is mixed to provide a solution substantially free of solids.

69-83. (canceled)

84. A method of treating a subject suffering from depression or postpartum depression, the method comprising administering the aqueous solution of claim 1, thereby treating a subject.
